SEQUENCE LISTING (38 sequences)

SEQ ID NO: 1 - protein, 13 aa, Rattus rattus
CARDVNFGNYFDY

SEQ ID NO: 2 - protein, 11 aa, Rattus rattus
CQQYSSWPPTF

SEQ ID NO: 3 - protein, 13 aa, Rattus rattus
CARDRGMGNYFDY

SEQ ID NO: 4 - protein, 11 aa, Rattus rattus [sequence omitted]

SEQ ID NO: 5 - protein, 15 aa, Rattus rattus
CARDATRILADVLDY

SEQ ID NO: 6 - protein, 10 aa, Rattus rattus
CQQYSSWYTF

SEQ ID NO: 7 - protein, 16 aa, Rattus rattus
CARAWRLSARAGYFDY

SEQ ID NO: 8 - protein, 11 aa, Rattus rattus [sequence omitted]

SEQ ID NO: 9 - protein, 11 aa, Rattus rattus
CILSDTGAFDV

SEQ ID NO: 10 - protein, 10 aa, Rattus rattus
CQQYSSWYTF

SEQ ID NO: 11 - protein, 12 aa, Rattus rattus
CTGAGPTYYFDY

SEQ ID NO: 12 - protein, 11 aa, Rattus rattus
CQQGYTTPVTF

SEQ ID NO: 13 - protein, 10 aa, Rattus rattus
RSQSHHHHHH

SEQ ID NO: 14 - DNA, 390 bp, Rattus rattus
caggtgcagctggtgcagtctggggcggaggtgaagaagcctggggcctccgtgaaggtctcctgcaaggcttctggatacaccttcaccagctatgctatcagctgggcgcgacagccccctggacaagggctcgagtggatgggagcttttgatcctgaatatggtagtacaacctacgcacagaagttccagggcagagtcaccatgaccgcagacacgtccacaagcacagcctatatggagctgagcagcctgagacctgaggacacggccgtgtattactgtgcgagagatgttaacttcggtaactattttgactactggggccaggggaccctggtcaccgtctcctcagcctccaccaagaacccagatgtcttccccctg

SEQ ID NO: 15 - protein, 130 aa, Rattus rattus
QVQLVQSGAEVKKPGASVKVSCKASGYTFTSYAISWARQPPGQGLEWMGAFDPEYGSTTYAQKFQGRVTMTADTSTSTAYMELSSLRPEDTAVYYCARDVNFGNYFDYWGQGTLVTVSSASTKNPDVFPL

SEQ ID NO: 16 - DNA, 390 bp, Rattus rattus
caggtgcagctggtgcagtctggggctgaggtgaagaagcctggggcctccgtgaaggtctcctgcaaggcttctggatacaccttcaccagctatgctatcagctgggtgcgacagccccctggacaagggctcgagtggatgggaggtgttgatcctgaatatggtggtacaacctacacacagaagttccagggcagagtcaccatgaccacagacacgtccacaagcacagcctacatggagctgagcagcctgagacctgaagacacggccgtgtattactgtgcgagagatcgcggtatggggaattactttgactactggggccaggggaccctggtcaccgtctcctcagcctccaccaagaacccagatgtcttccccctg

SEQ ID NO: 17 - protein, 130 aa, Rattus rattus
QVQLVQSGAEVKKPGASVKVSCKASGYTFTSYAISWVRQPPGQGLEWMGGVDPEYGGTTYTQKFQGRVTMTTDTSTSTAYMELSSLRPEDTAVYYCARDRGMGNYFDYWGQGTLVTVSSASTKNPDVFPL

SEQ ID NO: 18 - DNA, 390 bp, Rattus rattus
caggtgcagctggtgcagtctggggcggaggtgaagaagcctggggcctccgtgaaggtctcctgcaaggcttctggatacaccttcaccagctatggtatgcagtgggtgcgacaggcccctgaacaagggctcgagtggatgggatggatcaataccaacactggtggcacaagctacgcacagaagttccagggcagagtcaccatgaccagggacgcatccacgagtacagcctacatggagctgagcagcctgagacctgaggacacggccgtgtattactgtgcgagagatgcaacacgtatactagcggacgttcttgactactggggccaggggaccctggtcaccgtctcctcagcctccaccaagaacccagatgtcttc

SEQ ID NO: 19 - protein, 130 aa, Rattus rattus
QVQLVQSGAEVKKPGASVKVSCKASGYTFTSYGMQWVRQAPEQGLEWMGWINTNTGGTSYAQKFQGRVTMTRDASTSTAYMELSSLRPEDTAVYYCARDATRILADVLDYWGQGTLVTVSSASTKNPDVF

SEQ ID NO: 20 - DNA, 390 bp, Rattus rattus
caggtgcagctgcaggagtcagggggaggcttggttcagcccggggggtccctgagactctcctgtgcggcctctgaattcaccttcagtaactactacatgagctgggtccgccaggctccagggaaggggctggagtgggtctcatatattagttatgatggtggtagcacgtactacgcagactccgtgaagggccgattcaccatctccagagacaacgccaagaactcgctgtatctgcagatgaacagcctgagagccgaggacacggccgtgtattactgtgcgagagcgtggcggctgtcggctagagctgggtactttgactactggggccaggggaccctggtcaccgtctcctcagcctccaccaagaacccagatgtc

SEQ ID NO: 21 - protein, 130 aa, Rattus rattus
QVQLQESGGGLVQPGGSLRLSCAASEFTFSNYYMSWVRQAPGKGLEWVSYISYDGGSTYYADSVKGRFTISRDNAKNSLYLQMNSLRAEDTAVYYCARAWRLSARAGYFDYWGQGTLVTVSSASTKNPDV

SEQ ID NO: 22 - DNA, 390 bp, Rattus rattus
gaggtgcagctggtggagtctgggggaggcttggttcagcccggggggtccctgagactctcctgtgcggcctctggattcaccttcagtgactactacgtgaactgggtccgccagactccggggaagggcccagagtgggtaggttttattagaaacaaagccaatggtgggacagcggaatacgccgcgtctgtgaaaggccgattcaccatctcaagagatgattcaaagaactcgctgtatctgcaaatgagcggcctgaaaaccgaggacacggccgtatattactgtatactatcggatacgggcgcttttgatgtatggggccaagggaccatggtcaccgtctcttcagcctccaccaagaacccagatgtcttccccctg

SEQ ID NO: 23 - protein, 130 aa, Rattus rattus
EVQLVESGGGLVQPGGSLRLSCAASGFTFSDYYVNWVRQTPGKGPEWVGFIRNKANGGTAEYAASVKGRFTISRDDSKNSLYLQMSGLKTEDTAVYYCILSDTGAFDVWGQGTMVTVSSASTKNPDVFPL

SEQ ID NO: 24 - DNA, 390 bp, Rattus rattus
caggtgcagctgcaggagtcagggggaggcttggcaaagcctgggggttccctgagactcacctgtgcggcctctggattcaccttcagtgactactggatgagctgggtccgccaggctccagggaaggggttggagtgggttggagaaattaatcctgatgggggtagaacaaactacaaagacttcgtgaaaggccgattcaccatctccagagacaacgccaagaacacactttatctgcaattgaacagccttaaaaccgaggacacagccatctattactgtactggagctgggcccacatattactttgactactggggccaggggaccctggtcaccgtctcctcagcctccaccaagaacccagatgtcttccccctgaca

SEQ ID NO: 25 - protein, 130 aa, Rattus rattus
QVQLQESGGGLAKPGGSLRLTCAASGFTFSDYWMSWVRQAPGKGLEWVGEINPDGGRTNYKDFVKGRFTISRDNAKNTLYLQLNSLKTEDTAIYYCTGAGPTYYFDYWGQGTLVTVSSASTKNPDVFPLT

SEQ ID NO: 26 - DNA, 390 bp, Rattus rattus
gagctcgtgatgactcagtctccagccaccctgtctttgtctccaggggaaagagccaccgtctcctgcagggccggtcagagtgttagttactacttagcctggtaccagcagaaacctgggcaggctcccaggctcctcatctatggtgcatccaccagagccactggcatcccagccaggttcagcggcagtaggtctgggacagacttcactctcaccatcagcagcctggagcctgaagattttgcagtttattactgtcagcagtatagcagctggccacccactttcggccaagggaccaagctggagatcaaacgagctgtggctgcgccgtctgtcttcatcttcccgccatctgaggatcaggtgaaatctggaactgcc

SEQ ID NO: 27 - protein, 130 aa, Rattus rattus
ELVMTQSPATLSLSPGERATVSCRAGQSVSYYLAWYQQKPGQAPRLLIYGASTRATGIPARFSGSRSGTDFTLTISSLEPEDFAVYYCQQYSSWPPTFGQGTKLEIKRAVAAPSVFIFPPSEDQVKSGTA

SEQ ID NO: 28 - DNA, 390 bp, Rattus rattus
gagctcgtgatgactcagtctccagccaccctctctttgtctccaaaagaaacagccaccctctcctgcagggccagtcagagtgttagtagctacttagcctggtaccagcagaaacctgggcaggctcccaggctcctcatctatggtgcatccaccagagccactggcatcccagccaggttcagcggcagtgggtctgggacagacttcactctcaccatcagcagactggagcctgaagattttgcagtttattactgtcagcagtatagcagctggccactcactttcggccaagggaccaagctggagatcaaacgagctgtggctgcgccgtctgtcttcatcttcccgccatctgaggatcaggtgaaatctggaactgcc

SEQ ID NO: 29 - protein, 130 aa, Rattus rattus
ELVMTQSPATLSLSPKETATLSCRASQSVSSYLAWYQQKPGQAPRLLIYGASTRATGIPARFSGSGSGTDFTLTISRLEPEDFAVYYCQQYSSWPLTFGQGTKLEIKRAVAAPSVFIFPPSEDQVKSGTA

SEQ ID NO: 30 - DNA, 390 bp, Rattus rattus
gagctcacactcacgcagtctccagtcaccctctctttgtctccaaaagaaacagccaccctctcctgcagggccagtcagagtgttagaagctacttagcctggtaccagcagaaacctgggcaggctcccaggctcctcatctatggtgcatccaccagagccactggcatcccagccaggttcagcggcagtgggtctgggacagacttcactctcaccatcagcagcctggagcctgaagattttgcagtttattactgtcagcagtatagcagctggtacactttcggccaagggaccaagctggagatcaaacgagctgtggctgcgccgtctgtcttcatcttcccgccatctgaggatcaggtgaaatctggaactgccact

SEQ ID NO: 31 - protein, 130 aa, Rattus rattus
ELTLTQSPVTLSLSPKETATLSCRASQSVRSYLAWYQQKPGQAPRLLIYGASTRATGIPARFSGSGSGTDFTLTISSLEPEDFAVYYCQQYSSWYTFGQGTKLEIKRAVAAPSVFIFPPSEDQVKSGTAT

SEQ ID NO: 32 - DNA, 390 bp, Rattus rattus
  misc_feature (323)..(323): n is a, c, g, or t
gagctcacactcacgcagtctccatcctccctgtctgcatctgtaggagacagagtcaccatcacttgccgggcgagtcaggacattagaggttatttagcctggtatcaacagaaaccagggaaatctcctaggcttctgatctattctgcatctactttgcaaactggggttccctctcggttcagtggcagtagatctgggacagactacactctcaccatcagcagcctgcagtctgaagatgtggcaacttattattgtcaacagcattacagtactccactcactttcggccaagggaccaagctggagatcaaacnagctgtggctgcgccgtctgtcttcatcttcccgccatctgaggatcaggtgaaatctggaactgcc

SEQ ID NO: 33 - protein, 130 aa, Rattus rattus
  misc_feature (108)..(108): Xaa can be any naturally occurring amino acid
ELTLTQSPSSLSASVGDRVTITCRASQDIRGYLAWYQQKPGKSPRLLIYSASTLQTGVPSRFSGSRSGTDYTLTISSLQSEDVATYYCQQHYSTPLTFGQGTKLEIKXAVAAPSVFIFPPSEDQVKSGTA

SEQ ID NO: 34 - DNA, 390 bp, Rattus rattus
gagctcgtgatgacgcagtctccagccaccctctctttgtccccaaaagaaacagccaccctctcctgcagggccagtcagagtgttagaagctacttagcctggtaccagcagaaacctgggcaggctcccaggctcctcatctatggtgcatccaccagagccactggcataccagccaggttcagcggcagtgggtatgggacagacttcactctcaccatcagcagcctggagcctgaagattttgcagtttattactgtcagcagtatagcagctggtacactttcggccaagggaccaagctggagatcaaacgagctgtggctgcgccgactgtcttcatcttcccgacatctgaggatcaggtgaaatctggaactgccact

SEQ ID NO: 35 - protein, 130 aa, Rattus rattus
ELVMTQSPATLSLSPKETATLSCRASQSVRSYLAWYQQKPGQAPRLLIYGASTRATGIPARFSGSGYGTDFTLTISSLEPEDFAVYYCQQYSSWYTFGQGTKLEIKRAVAAPTVFIFPTSEDQVKSGTAT

SEQ ID NO: 36 - DNA, 390 bp, Rattus rattus
gagctcgtgatgactcagtctccatcctccctgtttgcatctataggagacagagtcaccattacttgccgggcgagtcagaacattagaagtaatttagcctggtatcaacagaaaccaggaaaaactcctaggctcctgatctatgatgcatctagtttgcaacctgggattccctctcggttcagtggcagtggatctgggacatattacactctcaccatcagcagcctgcagtctgatgatcttgccacttattactgtcaacaaggttatactactccagtcactttcggccaagggaccaagctggagatcaaacgagctgtggctgcgccgtctgtcttcatcttcccgccatctgaggatcaggtgaaatctggaactgcc

SEQ ID NO: 37 - protein, 130 aa, Rattus rattus
ELVMTQSPSSLFASIGDRVTITCRASQNIRSNLAWYQQKPGKTPRLLIYDASSLQPGIPSRFSGSGSGTYYTLTISSLQSDDLATYYCQQGYTTPVTFGQGTKLEIKRAVAAPSVFIFPPSEDQVKSGTA

SEQ ID NO: 38 - protein, 15 aa, Rattus rattus
WINPGRSRSHHHHHH

(19) United States
(12) Patent Application Publication
(10) Pub. No.: US 2005/0009096 A1
(43) Pub. Date: Jan. 13, 2005

(54) Method for diagnosis and prognosis of multiple sclerosis

(75) Inventors: Claude P. Genain, Mill Valley, CA (US); Hans-Christian Von Budingen, Schlier (DE); Til Menge, San Francisco, CA (US)

Correspondence Address: QUINE INTELLECTUAL PROPERTY LAW GROUP, P.C., P O BOX 458, ALAMEDA, CA, 94501, US

(73) Assignee: The Regents of the University of California

(21) Appl. No.: 10/683,451

(22) Filed: Oct. 10, 2003

Related U.S. Application Data
(60) Provisional application No. 60/418,001, filed on Oct. 11, 2002.

(51) Int. Cl. G01N 33/53

(52) U.S. Cl. 435/7.1

(57) ABSTRACT

This invention provides methods utilizing detection/quantification of autoantibodies to specific epitopes of myelin components (e.g. to conformational epitope of myelin/oligodendrocyte glycoprotein (MOG)) for the definitive diagnosis, and/or staging or typing, and/or prognosis of multiple sclerosis.

### CROSS-REFERENCE TO RELATED APPLICATIONS

This application claims benefit of and priority to U.S. Ser. No. 60/418,001, filed on Oct. 11, 2002, which is incorporated herein by reference in its entirety for all purposes.

### STATEMENT AS TO RIGHTS TO INVENTIONS MADE UNDER FEDERALLY SPONSORED RESEARCH AND DEVELOPMENT

This work was supported, in part, by Grant Nos: 3320-A-3 and 3438-A-7 from the National Institutes of Health. The Government of the United States of America may have certain rights in this invention.

### FIELD OF THE INVENTION

This invention pertains multiple sclerosis. In particular this invention provides improved diagnostics and prognostics for diagnosing, staging, or predicting outcome for a patient having multiple sclerosis.

### BACKGROUND OF THE INVENTION

Multiple sclerosis (MS) designates a group of heterogeneous, immune-mediated demyelinating disorders of the central nervous system (CNS). There is currently no paraclinical investigation that accurately predicts clinical course, prognosis, or pathological subtypes for individual patients. MS pathogenesis is complex and multifactorial, with a strong genetic component likely acting in concert with environmental exposure(s). In addition to the well-established major histocompatibility complex association, recent full genome screening of MS families supports the role of several unidentified genes each with modest effect. Previous studies support the hypothesis that humoral immunity plays a role in MS pathogenesis, and the heightened incidence of antibodies associated with autoimmune disorders observed in MS families suggest that genetic factors may control susceptibility to develop humoral autoimmunity. Anti-myelin antibodies are of particular importance to study in MS, since they reflect CNS-specific humoral responses.

Whereas mediation of tissue damage by anti-myelin antibodies can be unequivocally demonstrated in the disease model experimental allergic encephalomyelitis (EAE), the pathophysiological significance of such antibodies in humans is uncertain because they are frequently detected in sera of both MS-affected and control subjects. This may indicate that regulatory mechanisms that would normally prevent autoantibodies to gain access to the CNS, for example suppressive T cell responses, are defective in MS patients. An additional possible explanation is that autoantibodies to CNS constituents are functionally heterogeneous, and qualitatively differ in MS from that in other subjects as a result of genetic polymorphisms and/or exposure of the immune system to antigenic determinants specifically associated with pathogenicity. Unlike T helper cells responses, which require antigen processing and presentation and are thus restricted to short antigenic peptides, antibodies most often target additional determinants on proteins that are defined by their tertiary structure. Studies of antibody repertoire specificity that account for the complexity of humoral responses in outbred populations are needed in order to elucidate their pathogenic properties in disorders like MS.

### SUMMARY OF THE INVENTION

Using combinatorial Fab fragments libraries, we have characterized the diversity of autoantibody responses against myelin/oligodendrocyte glycoprotein (MOG) during EAE in the C. jacchus marmoset, a non-human primate in which pathogenic autoantibodies are obligatory for the formation of MS-like demyelinating plaques. Several discrete, tertiary structure-dependent determinants have been defined on the surface-exposed, extracellular domain of MOG, and distinct populations of native polyclonal antibodies have been characterized based on their ability to bind to short linear peptides or structurally defined epitopes of MOG. Our results strongly suggest that pathogenicity is correlated with recognition of the structural determinants of MOG, in agreement with previous studies of murine monoclonal antibodies.

Directly relevant to MS pathophysiology, we found that conformational epitopes recognized by marmoset antibodies appear commonly within the anti-MOG antibody repertoires of MS sera. Recombinant C. jacchus Fab fragments that define structural epitopes of MOG recognized by autoantibodies in humans now afford refined studies of pathogenic autoantibody responses in MS.

Thus, without being bound to a particular theory, we believe that development of pathogenic humoral immunity in MS is controlled at least in part at the genomic level, and that comprehensive serum autoantibody profiling in MS families defines distinct clinical phenotypes.

Thus, in various embodiments, this invention contemplates methods utilizing detection/quantification of autoantibodies to specific epitopes of myelin components (e.g. to conformational epitope of myelin/oligodendrocyte glycoprotein (MOG)) for the definitive diagnosis, and/or staging or typing, and/or prognosis of multiple sclerosis.

Thus, in one embodiment, this invention provides a method of diagnosing or evaluating the prognosis of multiple sclerosis (MS) or allergic encephalomyelitis (EAE) in a mammal. The method typically involves detecting the presence or quantity of an antibody in the mammal specific for a conformational epitope of myelin/oligodendrocyte glycoprotein (MOG) where the presence or increased concentration of the antibodies indicates the presence of a particular stage of multiple sclerosis or the increased likelihood of the development of a more severe form of the disease. In certain embodiments, the detecting comprises obtaining a biological sample comprising serum or cerebrospinal fluid from the mammal. In certain embodiments, can involve screening for a plurality of antibodies specific for different conformational epitopes of the myelin/oligodendrocyte glycoprotein. In certain embodiments, the antibody specific for a conformational epitope of myelin/oligodendrocyte glycoprotein is an antibody that specifically binds to an epitope specifically bound by an antibody comprising a polypeptide sequence selected from the group consisting of SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, and SEQ ID NO:37. The detecting can, optionally involve a competitive assay using a competitive binder an antibody comprising a CDR3 comprising a peptide sequence as shown in Table 2 (SEQ ID NOs:1-12). In certain embodiments, the detecting involves a competitive assay using as a competitive binder an antibody specific for a conformational epitope of myelin/oligodendrocyte glycoprotein is an antibody that specifically binds to an epitope bound by an antibody comprising a polypeptide sequence selected from the group consisting of SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, and SEQ ID NO:37. In certain embodiments, the detecting comprises a competitive assay using as a competitive binder an antibody specific for a conformational epitope of myelin/oligodendrocyte glycoprotein where the antibody comprises a polypeptide sequence selected from the group consisting of SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, and SEQ ID NO:37. The mammal can be a human (e.g. a human with a preliminary diagnosis of multiple sclerosis) or a non-human mammal.

In another embodiment, this invention provides a method of evaluating the risk of progressing to a severe form of multiple sclerosis and/or the extent of central nervous system damage in a mammal. The method typically involves obtaining a biological sample comprising serum or cerebrospinal fluid from the mammal; and detecting the proportion of autoantibodies specific for a conformational epitope to those specific for a linear MOG epitope or a linear epitope of another myelin protein; where an increased ratio of conformational specific antibodies indicates an increased likelihood or progressing to a severe form of the disease and/or increased central nervous system damage. In certain embodiments, detecting the proportion comprises detecting binding of autoantibodies to a MOG conformational epitope and to a MOG linear peptide. In certain embodiments, detecting the proportion comprises determining the ratio of MOG-peptide-specific to rMOG-specific antibodies. In certain embodiments, the detecting comprises screening for a plurality of antibodies specific for different conformational epitopes of the myelin/oligodendrocyte glycoprotein. The antibodies specific for a conformational epitope of myelin/oligodendrocyte glycoprotein include, but are not limited to an antibody that specifically binds to an epitope bound by an antibody comprising a polypeptide sequence selected from the group consisting of SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, and SEQ ID NO:37. In certain embodiments, the detecting comprises a competitive assay using a competitive binder an antibody comprising a CDR3 comprising a peptide sequence as shown in Table 2 (SEQ ID NOs:1-12). In certain embodiments, the detecting comprises a competitive assay using as a competitive binder an antibody specific for a conformational epitope of myelin/oligodendrocyte glycoprotein is an antibody that specifically binds to an epitope bound by an antibody comprising a polypeptide sequence selected from the group consisting of SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, and SEQ ID NO:37. In certain embodiments, the detecting comprises a competitive assay using as a competitive binder an antibody specific for a conformational epitope of myelin/oligodendrocyte glycoprotein where the antibody comprises a polypeptide sequence selected from the group consisting of SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, and SEQ ID NO:37. The mammal can be a human (e.g. a human with a preliminary diagnosis of multiple sclerosis) or a non-human mammal (e.g. a test/model animal).

This invention also provides a method of treating a patient having a preliminary diagnosis of multiple sclerosis. The method typically involves obtaining a biological sample comprising serum from the patient; and detecting autoantibodies specific for a conformational epitope to those specific for a linear MOG epitope or a linear epitope of another myelin protein; and prescribing a more aggressive treatment regimen when the ratio is elevated (e.g. as compared to that observed in healthy patients and/or in patients having a mild or non-progressive form of the disease).

Also provided is a method of diagnosing definite multiple sclerosis in patients with a first episode of demyelination in the central nervous system. The method typically involves measuring antibodies against specific myelin constituents where the presence and/or quantity of such antibodies indicates a definite diagnosis of multiple sclerosis. In certain embodiments, the myelin constituent comprises MOG and/or Galc. In certain embodiments, the antibodies are specific for a conformational epitope of MOG and/or a conformational epitope of Galc.

In still another embodiment this invention provides a method of determining the form of multiple sclerosis. The method typically involves measuring a plurality of antibodies against specific myelin constituents where presence or level of certain members of the plurality indicate the form or stage of multiple sclerosis. In certain embodiments, the myelin constituent comprises MOG and/or Galc. In certain embodiments, the detecting comprises detecting the presence or quantity of an antibody specific for a conformational epitope of myelin/oligodendrocyte glycoprotein (MOG). The detecting can comprise screening for a plurality of antibodies specific for different conformational epitopes of the myelin/oligodendrocyte glycoprotein and/or Galc. In certain embodiments, the antibody specific for a conformational epitope of myelin/oligodendrocyte glycoprotein is an antibody that specifically binds to an epitope bound by an antibody comprising a polypeptide sequence selected from the group consisting of SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, and SEQ ID NO:37. In certain embodiments, the detecting comprises a competitive assay using a competitive binder an antibody comprising a CDR3 comprising a peptide sequence as shown in Table 2 (SEQ ID NOs:1-12). In certain embodiments, the detecting comprises a competitive assay using as a competitive binder an antibody specific for a conformational epitope of myelin/oligodendrocyte glycoprotein is an antibody that specifically binds to an epitope bound by an antibody comprising a polypeptide sequence selected from the group consisting of SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, and SEQ ID NO:37. In certain embodiments, the detecting comprises a competitive assay using as a competitive binder an antibody specific for a conformational epitope of myelin/oligodendrocyte glycoprotein where the antibody comprises a polypeptide sequence selected from the group consisting of SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, and SEQ ID NO:37.

In certain embodiments, this invention provides a method of predicting disease outcome in patients with a first episode of demyelination in the central nervous system or with definitive multiple sclerosis. The method typically involves measuring antibodies against specific myelin constituents where the presence or increasing concentrations of such antibodies indicates a progressively negative outcome. In certain embodiments, the myelin constituent comprises MOG and/or Galc. In certain embodiments, the antibodies are specific for a conformational epitope of MOG and/or Galc. The method can, optionally, involve measuring the antibodies at two or more times. In certain embodiments, the two or more times comprises a first time at initial presentation or diagnosis of the disease and a second time at least two months later.

This invention also provides methods of estimating the time within the history of an individual patient when MS disease will transform from benign to progressive. The methods typically involve measuring a plurality of antibodies against specific myelin constituents where presence or level of certain members of the plurality indicate the imminence of transformation of MS from benign form to a progressive form. In certain embodiments, the myelin constituent comprises MOG and/or Galc. In certain embodiments, the measuring comprises detecting the presence or quantity of an antibody specific for a conformational epitope of myelin/oligodendrocyte glycoprotein (MOG). In certain embodiments, the measuring comprises screening for a plurality of antibodies specific for different conformational epitopes of the myelin/oligodendrocyte glycoprotein. In certain embodiments, the antibody specific for a conformational epitope of myelin/oligodendrocyte glycoprotein is an antibody that specifically binds to an epitope bound by an antibody comprising a polypeptide sequence selected from the group consisting of SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, and SEQ ID NO:37. In certain embodiments, the method involves measuring the antibodies at two or more times. In certain embodiments, the two or more times comprises a first time at initial presentation or diagnosis of the disease and a second time at least two months later.

Also provided are recombinant proteins consisting essentially of a MOG extracellular domain and a truncation at the C-terminus, wherein the protein is soluble in an aquous buffer at neutral pH. In certain embodiments, the protein is a protein selected from the group consisting of Rat MOG 1-117, Rat MOG 1-125, human MOG 1-118, and human MOG 1-125.

In addition, this invention provides an assay for detecting antibodies to conformational epitopes of MOG in a mammal. The assay typically involves providing a serum or CSF sample from the subject; and contacting antibodies in the sample with two or more recombinant proteins as described herein where specific binding of one or more of the recombinant proteins to the antibodies indicates the presence of one or more antibodies antibodies to conformational epitopes of MOG in the mammal. In certain embodiments, the two or more proteins are independently selected from the group consisting of Rat MOG 1-117, Rat MOG 1-125, human MOG 1-118, and human MOG 1-125.

Definitions

The terms “polypeptide”, “peptide” and “protein” are used interchangeably herein to refer to a polymer of amino acid residues. The terms apply to amino acid polymers in which one or more amino acid residues is an artificial chemical analogue of a corresponding naturally occurring amino acid, as well as to naturally occurring amino acid polymers.

The term “antibody” refers to a polypeptide substantially encoded by an immunoglobulin gene or immunoglobulin genes, or fragments thereof which specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially an Fab with part of the hinge region (see, Fundamental Immunology, Third Edition, W. E. Paul, ed., Raven Press, N. Y. 1993). While various antibody fragments are defined in terms of the digestion of an intact antibody, one of skill will appreciate that such fragments may be synthesized de novo either chemically or by utilizing recombinant DNA methodology. Thus, the term antibody, as used herein, also includes antibody fragments either produced by the modification of whole antibodies, those synthesized de novo using recombinant DNA methodologies (e.g., single chain Fv), and those found in display libraries (e.g. phage display libraries).

The term “specifically binds”, as used herein, when referring to a binding agent (e.g., protein, nucleic acid, antibody, etc.), refers to a binding reaction that is determinative of the presence binding agent in a heterogeneous population of proteins and other biologics. Thus, under designated conditions (e.g. immunoassay conditions in the case of an antibody, or stringent hybridization conditions in the case of a nucleic acid), the specified ligand or antibody binds to its particular “target” (e.g. a protein or nucleic acid) and does not bind in a significant amount to other molecules.

The term a “conformational epitope”, e.g. when referring to a conformation epitope of a MOG, refers to region of the subject protein that is specifically recognized by an antibody and that introduces secondary or tertiary structure into the subject protein. This is as distinguished from “linear epitope” that refers to a region of the protein that does not introduce secondary structure (e.g. bends, helices, etc.). A conformational epitope can be identified by any of a number of methods known to one of skill in the art. For example, when a conformational epitope is “denatured” i.e. the conformation is altered and/or linearized, binding by the conformational epitope specific antibody is diminished or eliminated. In contrast, “denaturation” of a linear epitope will not substantially alter binding by antibodies specific to that epitope.

A “MOG conformational epitope antibody” refers to an antibody that specifically binds a conformational epitope of a MOG protein.

### BRIEF DESCRIPTION OF THE DRAWINGS

FIG. 1. illustrates the conformational requirements of MOG-specific Fab clones. Means of triplicate or quadruplicate values. (*) PepMOG designates a mixture of overlapping 20-mer peptides spanning the entire sequence of rMOG. For comparison, representative reactivity of rMOG-immune serum Abs is also shown (Serum).

FIGS. 2A, 2B, and 2C illustrate the results of competition ELISAs with representative Fab fragments. FIG. 2A) M26Biotin is displaced from rMOG by itself (O),M38 (⋄), and M45(X) but not by M3-8 (□), M3-24 (Δ), or M3-31 (inverted triangle). (FIG. 2B) M3-24Biotin is displaced only by itself (Δ). (FIG. 2C) M3-31Biotin is displaced by itself (inverted triangle), M38 (⋄), and M45 (X), and also by high concentrations of M26 (O). Highlighted in red are the Fab fragments that tightly cluster within the major immunogenic region of MOG. IGHV and IGKV gene usage is indicated in the legend.

FIG. 3 illustrates binding of recombinant Fab fragments to MOG in situ on C. jacchus CNS myelin. Fluorescent light micrographs of C. jacchus corpus callosum showing oligodendrocytes and myelinated fibers stained with the biotinylated Fab fragment M26 (Left). Specificity of the staining was confirmed by signal quenching after coincubation with rMOG (Right). Arrows indicate groups of aligned oligodendrocyte cell bodies.

FIG. 4 shows correlations between anti-MOG Ab epitope recognition and neuropathological phenotypes. (Upper) Perivascular mononuclear cell infiltrates in brain white matter of representative rMOG-(Left) and PepMOG-immunized marmosets (Right). Note the large size of the infiltrate and the broad area of demyelination in the rMOG-immunized animal, and the lack of demyelination after PepMOG-immunization. Luxol Fast Blue/periodic acid Schiff, ×200. (Lower) The specificities of serum anti-MOG Abs in these animals were analyzed before (Serum) and after (PepMOG-depleted) removal of Abs binding to PepMOG (see Materials and Methods in Examples). rMOG reactivity is clearly retained after removal of the peptide-reactive Abs from rMOG-immune serum (Left), indicating the presence of separate subsets of Abs that react either with linear peptides or conformational rMOG. In marked contrast, no reactivity to MOG remains after removal of PepMOG-specific Abs from the animal immunized with PepMOG (Right), demonstrating that conformation-dependent Abs were not produced. Identical results were obtained in the other rMOG- and PepMOG-immunized monkeys.

FIG. 5 shows the results of competition between marmoset Fab fragments and human anti-MOG Abs. Affinity-purified serum anti-MOG Abs from patient AA with MS are displaced by M3-8 (□) and M3-24 (Δ) or a combination of and M3-8 and M3-24 (▾).

FIG. 6 illustrates neuropathology of EAE induced in C. jacchus by active immunization with whole rMOG (top), MOG aa21-40 (middle), and adoptive transfer of a MOG aa20-40-reactive T cell clone (bottom).

FIG. 7 shows lesion load in the entire neuraxis (brain, optic nerves and spinal cord) of MOG-peptide and rMOG-immunized marmosets, respectively (mean±SD).

FIGS. 8A and 8B illustrate fractionation of MOG-specific serum Ig by affinity-chromatography. Ig binding to MOG-peptides was removed from serum using MOG peptide-Sepharose columns, and acid eluted. F low through fractions (depleted of all Ig binding to MOG-peptides), and eluted fractions (containing the peptide-binding Ig) were tested by ELISA for IgG reactivity to rMOG and MOG-peptides, respectively (insets). FIG. 8A: rMOG-immune serum: reactivity to rMOG is still detected after removal of peptide-binding IgG, indicating the presence of IgG binding to strictly conformational determinants (red). Note that the MOG peptide binding IgG also recognize rMOG (blue). FIG. 8B: MOG-peptide immune serum: removal of peptide-binding IgG results in the complete loss of reactivity to rMOG.

FIG. 9 illustrates the detection of macrophages (HAM56), IgG and activated complement (C9neo) by immunostaining of EAE lesions from rMOG-(n=4) and MOG peptide-immunized marmosets (n=9). IgG cells designate cells positively stained for IgG, likely plasmocytes. A total of 84 lesions were examined and the percentage of positive lesions is shown. Data are Mean ±SEM.

FIG. 10 illustrates competition of Fab fragments against native anti-MOG IgG, and the 8.18.C5 antibody. Constant concentrations of biotinylated, purified C. jacchus anti-MOG IgG (animal 318-97) were incubated with increasing concentrations of individual Fab fragments (red, blue), combination of both Fabs (green), or the non-biotinylated anti-MOG IgG themselves (black diamonds). Y-axis: % of MOG-bound biotinylated IgG. X-axis: log of concentration of competitor. In contrast to C. jacchus Fabs, 8.18.C5 fails to compete with purified marmoset anti-MOG IgG (black circles).

FIG. 11 shows the staining of MOG-transfected COS cells (top panel) and fibroblast cell line CCL-153 (middle panel) with biotinylated M26 Fab. Rightpanel: an untransfected cell line.

FIG. 12 illustrates the transfer of human IgG in MBP-immunized marmosets. Top panel: transfer of IgG from an MS serum reactive to MOG. Large subpial infiltrate with underlying demyelination in the spinal cord (LFB/PAS). Bottom panel: spinal cord of an animal transferred with IgG from a control, unreactive serum. Subpial infiltrate with intact underlying myelin (H&E).

FIG. 13 shows the percentage of sera testing positive for MOG and MBP antibody in the different clinical phenotypes of MS. The number of patients studied for each MS subtype is given in parentheses on the X axis (MBP reactivity was assessed in only 17 of the controls). Results were replicated independently by 2 different technicians in the laboratory.

FIG. 14 shows serum reactivity (IgG) to rMOG, MBP, and MOG-derived 20 mer peptides in patient CIS 5 presenting with transverse myelitis, positive brain and cervical spine MRI, and Gd+ enhancement. Note the lack of reactivity to MBP in this patient.

FIG. 15 Anti-Galc antibody ELISA. Top panel, validation using marmoset sera: from left to right in succession, naïve control, animals immunized with adjuvant's mixture alone (CFA), rMOG (all negative), and time course of appearance of anti-Galc IgG in animals immunized with whole white matter. The animal with very high titers (*) had chronic EAE and was sampled after 3 relapses. A rabbit polyclonal anti-Galc antibody is used as positive control (far right). Bottom panel, human sera from 6 individual patients with MS. Sera were diluted 1:100. Results are means of duplicate wells, corrected for background values for each patient, which ranged from 0.05 to 0.12 OD units.

FIG. 16A shows sequential studies of IgG reactivity to MOG, MBP and Galc in patient DM. FIG. 16B shows time-dependent variation in titers and epitope recognition of rMOG-specific IgG in a patient with SPMS. Note the low of reactivity to MBP. Results are for sera diluted 1:100 and background corrected. Serial measurements for each patient were performed in a single assay plate

FIG. 17 illustrates fractionation of MOG-specific antibodies. C designates the fraction containing conformation-dependent antibodies, and L the fraction containing antibodies that recognize linear MOG peptides.

FIG. 18 shows the inverse correlation between the ratio of MOG/peptide-(AbPep) to rMOG-reactive IgG, and clinical severity of MOG-induced marmoset EAE (marmoset expanded scale, 0-45 points 82). Antibody measurements were performed quantitatively using serial serum dilutions and a standard curve for marmoset IgG.

FIG. 19 shows the results of passive transfers in MBP-immunized marmosets. Left, large confluent demyelinating infiltrates in a recipient of peptide-depleted, rMOG-purified Ig. Right, typical lesion in a recipient of MOG-peptide-specific Ig. Note minimal demyelination. LFB/PAS.

FIG. 20, panels A and B, show neuropathology of rMOG1-125- and MOG peptide-induced EAE. High power views (×200) of cervical spinal cord sections stained with LFB. Panel A: typical inflammatory infiltrate in a marmoset immunized with MOG aa21-40 (368-94). Note contiguity with the subpial space (upper right corner) and the limited amount of demyelination. Panel B: perivascular, inflammatory infiltrate in deep periventricular white matter (V=blood vessel) with pronounced concentric demyelination, characteristic of rMOG₁₋₁₂₅-immunized animals (J2-97). Such lesions were never found in MOG peptideimmune C. jacchus. Note myelin vacuolation (arrows) in both MOG peptide-induced EAE (A) and at the lesion edges in rMOG1-125-induced EAE (B).

FIG. 21, panels A through A show fine specificities of unfractionated sera and anti-MOG-P-depleted sera from representative animals of groups I and II. The left panels show reactivity of whole sera at a dilution of 1:200. The right panels show residual reactivity after removal of anti-MOG-P antibodies by affinity-chromatography. Panels A and B: Antibody specificities in rMOG₁₋₁₂₅-immunized monkeys (n=4, mean +/−SEM), demonstrating that strong reactivity against rMOG1-125 is retained after removal of all MOG peptide-specific antibodies. Panels C-F: Representative experiments for individual animals immunized with individual or all MOG-derived peptides (aa21-40, 199-94; pepMOG, 39-95). Panels G and H: reactivity of a pool of MOG peptide-immune sera (animals 252-93, Tx245-90, 14-91, Tx75-92, Tx256-93): The MOG-reactivity is completely removed in all animals immunized with MOG-derived peptides by passage on pepMOG columns, indicating that this immunization regimen does not induce conformation-dependent antibodies. Compare to A and B, rMOG1-125-immune animal.

FIG. 22, panels A through D show reactivity of affinity purified anti-MOG antibody fractions with native MOG. Immunohistochemical staining (brown) of normal brain tissue from an unimmunized C. jacchus. Panels A and B: anti-MOG-C and anti-MOG-P from an rMOG₁₋₁₂₅immune serum pool; Panel C: anti-MOG-P from a MOG peptide-immune serum pool; Panel D: naïve C. jacchus serum. Consecutive sections showing corpus callosum (cc) and adjacent gray matter (gm) at 200× magnification.

FIG. 23 shows T cell proliferation against _{rMOG1-125} ⁱⁿ _{rMOG1-125-} and MOG peptideimmune animals. Mean +/−SEM.

FIG. 24, panels A through F show immunohistochemical characterization of CNS lesions. Representative lesions from an rMOG₁₋₁₂₅-immunized animal (J2-97, left) and an animal immunized with MOG aa21-40 (199-94, right). From top to bottom, staining (brown) for macrophages (HAM56, Panels A and B); IgG (Panels C and D); C9neo (Panels E and F). IgG depositions were predominantly found in rMOG₁₋₁₂₅-immunized animals (Panel C) compared to MOG peptideimmune animals (Panel D). Activation of complement (C9neo) was a characteristic of rMOG₁₋₁₂₅-induced EAE (Panel E) and was not found in MOG peptide-immune animals (Panel F). Original magnification 600×.

FIG. 25 shows alignment of human, marmoset, and rat MOG.

### DETAILED DESCRIPTION

This invention pertains to diagnostics and prognostics for evaluation and/or treatment of multiple sclerosis. Human multiple sclerosis (MS) and the related disease model experimental allergic encephalomyelitis (EAE) are autoimmune disorders of the central nervous system characterized by destruction of myelin and axons. Antibodies to myelin are known to occur in multiple sclerosis.

Antibodies against certain myelin constituents, including myelin oligodendrocyte glycoprotein (MOG), and galactocerebroside (Galc), directly create myelin damage in experimental allergic encephalomyelitis (EAE) models. These antibodies, and others as well, can be detected in serum and cerebrospinal fluid of animals with EAE, and MS patients using established techniques, for example ELISA. However, because these techniques also detect antibodies in control subjects simple screening for, e.g. anti-MOG antibodies appeared to offer little diagnostic and/or prognostic value.

It was a surprising discovery of this invention that there exist certain classes of myelin autoantibodies in serum and cerebrospinal fluid and that the presence and/or quantity of these classes of antibodies is strongly correlated with the severe and progressive forms of multiple sclerosis. Accordingly this invention provides sensitive and specific assays (e.g. ELISA) systems to measure these antibodies and these assays provide effective diagnostics and/or prognostics for MS.

In particular, it was determined that autoantibodies against MOG are more frequently detected in severe, chronic progressive forms of MS. In primary progressive forms, incidence approximates 100% whereas in beginning MS, it is around 30-40%. Autoantibodies against MOG are present in a significant proportion (50%) of patients during a first clinical attack corresponding to a demyelinating event (clinically isolated syndrome, which does not meet criteria for a diagnostic of MS). These antibodies persist in worsening forms of MS, but decrease or disappear with improvement or stabilization. They also appear to disappear following treatment with disease modifying therapies (all unpublished data). It is believed that these dynamic patterns of specific anti-myelin antibodies in MS have not been previously recognized, and are not observed in the case of antibodies directed against another antigen of myelin, myelin basic protein.

In addition, we have discovered that autoantibodies against MOG segregate into several categories according to epitope recognition, including epitopes that are strictly conformational, and epitopes corresponding to linear, short peptides. Autoantibodies against conformational epitopes of MOG, and not those against linear peptides, are pathogenic in the marmoset model of EAE. The severity of EAE correlates with titers of autoantibodies against conformational epitopes of MOG, and not the titers of antibodies directed against linear peptides.

Autoantibodies to MOG in humans also segregate into strictly conformational, and linear peptide dependent classes.

In addition, it was discovered that autoantibodies against Galc appear late in the course of EAE in marmosets, and are also associated with chronic disease.

In one embodiment, this invention provides methods that involve measuring autoantibodies against MOG that have specificity restricted to conformational determinants of this protein in human. This was possible because we isolated antibody clones that represent these specificities and are able to use them as reagents in specific competition ELISA systems. The presence and/or level of such autoantibodies indicate the presence and/or prognosis and/or stage of multiple sclerosis.

This invention also provides methods that involve measuring the proportions of antibodies against conformational MOG epitopes and of those against the linear epitopes, or of those against other proteins. These methods are useful to assess the risk of developing severe forms of MS and/or the extent of central nervous system tissue damage (brain atrophy). This can be accomplished practically in ELISA (or other assay) systems that do not require physical separation of the different classes of antibodies. Such assays have direct application to prognosis and clinical management of MS patients.

In other embodiments, this invention contemplates methods that involve detecting antibodies against myelin constituents, including, but not limited to MOG, Galc, and other antigens in the blood and/or cerebrospinal fluid, for example, at regular intervals (e.g. initial presentation/diagnosis of the disease, at least one month later, at least 2 months later, at least 3, 4, or 6 months later), in order to: 1) Help diagnose definite MS in patients with a first episode of demyelination in the central nervous system. 2) Predict disease outcome for such patients, and also for patients with definite MS. 3) Help define the time within the history of individual patients when MS disease will transform from benign to progressive, severe forms which corresponds to major disability and brain atrophy. and 4) Diagnose the primary progressive forms of MS, when diagnosis cannot be ascertained by other means of evaluation (e.g., clinical, electrophysiological, MRI, standard cerebrospinal fluid studies, or others).

It was a discovery of ours that two or more repetitive tests can help physicians in the diagnosis, prognosis, and therefore therapeutic management, of MS patients. We also discovered that the presence and persistence of certain antibodies to myelin (for example, conformation-dependent binding antibodies as assessed by specific competition with newly developed recombinant marmoset Fab fragments), or antibody associations (one, two, or more), represent new means for clinicians to assess the risk of individual patients to develop severe MS. Furthermore, we believe such measurements can be used in MS as paraclinical marker(s) of tissue destruction (myelin damage, axonal loss, scarring), as suggested by the findings of high prevalence and persistence in severe forms of disease.

One particular relevant clinical index is described in Example 3. As described therein, the ratio of MOG-peptide-specific over rMOG-specific antibodies is predictive of the severity of clinical EAE in the marmoset. Thus it appears to be an extremely useful index for evaluating MS patients.

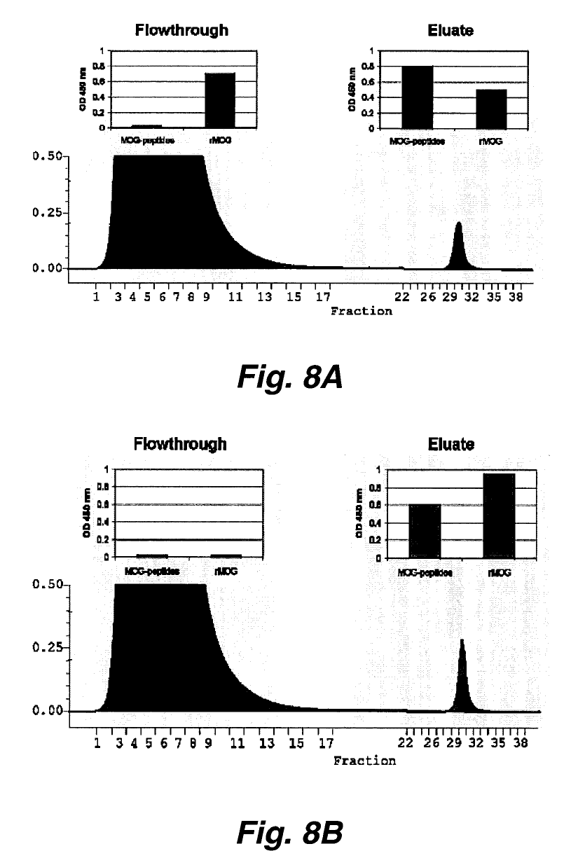

As illustrated in FIG. 8, it can be difficult to distinguish these different antibody fractions by ELISA where the ligand (MOG peptide) is attached to a substrate. The difference in epitope recognition, however, is important and translates into functional heterogeneity (e.g., pathogenic potential), since marmosets immunized with the linear peptides develop an attenuated EAE phenotype compared to rMOG-immunized animals, despite the apparent induction of similar T cell responses. We have also observed that disease severity in rMOG-induced marmoset EAE is inversely proportional to the ratio of serum concentrations (μg/ml) of MOG peptide/rMOG-reactive IgG.

Diatonistics/Prognostics for MS

As indicated above, it was a discovery of this invention that anti-MOG autoantibodies and/or anti-GALC antibodies, more preferably antibodies directed agains the conformation epitope(s) of MOG are particularly useful as measures of existence and/or stage and/or prognosis of multiple sclerosis in a mammal (e.g. a human or a non-human mammal).

Thus in various embodiments, this invention provides diagnostic and/or prognostic assays for multiple sclerosis that involve detecting and/or quantifying antibodies directed against (specific to) one or more epitopes of MOG and/or GALC, more preferably detecing antibodies specific to one or more conformational epitopes of MOG.

Typically the methods involve providing a biological sample from the mammal (e.g. human) that is to be screened. The biological sample is one that would typically be expected to contain anti-MOG antibodies (e.g. cerebrospinal fluid, blood, or blood fractions (e.g. serum). The sample can be “acute” or processed (e.g. diluted, fractionated, etc.). The sample is then screened for the presence and/or quantity/concentration of one or more of the antibodies in question (e.g. MOG conformational epitope antibodies).

Any of a variety of methods can be used to identify/quantify the antibodies in question. Such methods include electrophoretic methods, mass spectrometric methods, various immunoassays, and the like. Thus, for example, the target antibodies (e.g. MOG structural epitope antibodies) can be identified by fractionation methods, e.g. using affinity columns as described in Example 2.

In certain embodiments, any of a number of well recognized immunological binding assays (see, e.g., U.S. Pat. Nos. 4,366,241; 4,376,110; 4,517,288; and 4,837,168) are well suited to detection or quantification of the antibodies identified herein. For a review of the general immunoassays, see also Asai (1993) Methods in Cell Biology Volume 37: Antibodies in Cell Biology, Academic Press, Inc. New York; Stites & Terr (1991) Basic and Clinical Immunology 7th Edition.

Where it is desired to specifically detect conformational epitopes, assays that preserve the conformational epitope(s) of the protein are preferred. In “solid phase” ELISA systems, antigens may be “nonspecifically” bound to the plate and their structure or appearance may be altered to some extent. Thus, in certain preferred embodiments, a liquid phase assay is utilized. For example, we have created a liquid phase assay employing biotinylated MOG₁₋₁₁₈, MOG₁₋₁₂₅, MBP, and MOG peptides; after incubation of these antigens with serum, antibodies are captured by Protein G and immunocomplexed antigen detected by streptavidin, and/or other methods, of antibody capture such as protein L, anti-Fc, protein A/G coupled to agarose or sepaharose, etc.

In immunized marmosets specific antibodies can be detected readily, whereas in humans the frequencies appear lower than that observed with a classical ELISA. Without being bound to a particular theory, it is believed that the liquid phase assay only detects certain subgroups of antibodies (for example those with higher affinity and those reactive to conformational, but not linear epitopes). However, in view of the enhanced specificity for conformational epitopes, these assays will greatly enhance the interpretation of antibody profiling studies in patients with MS and clinically isolated syndromes (CIS, first clinically detectable demyelinating event).

We have generated a panel of novel recombinant proteins (e.g., rat MOG₁₋₁₁₇, rat MOG₁₋₁₂₅, human MOG₁₋₁₁₈, human MOG₁₋₁₂₅), that correspond to rat and human MOG extracellular domains with various truncations at the C-terminus. These proteins are soluble at mg/ml concentrations in aqueous buffers at neutral pH, unlike various previously available proteins. Most important, combined use of these recombinant MOG “variants” permits direct, one-step identification of epitope specificities that correspond to the conformational epitopes of MOG within the primate and human polyclonal repertoires (e.g., this avoids fractionation steps) (see, e.g., Table 1).

TABLE 1
Identification of structural target epitopes using
the recombinant MOG variants.

| | Epitope | | |
|---|---|---|---|
| Modified MOG peptide | M3-8 | M3-24 | M26 |
| Rat MOG 1-117 | + | − | + |
| Rat MOG 1-125 | + | + | + |
| Human MOG 1-118 | − | − | + |
| Human MOG 1-125 | − | + | + |

In certain embodiments, the anti-MOG and/or anti-Galc antibodies can be detected using protein and/or lipid/glycolipid microarrays comprising a plurality of MOG and/or Galc epitopes. Such arrays provide a powerful technique to allow allow one-step characterization of many antibody specificities (see, e.g., Robinson et al. (2002) Biotechniques Dec Suppl: 66-69; Liotta et al. (2003) Cancer Cell 3(4): 317-325; Bacarese et al. (2002) Biotechniques Dec Suppl: 24-9; Delechanty and Ligler (2003) Biotechniques 34(2): 380-385, and the like). Such methods are particularly suitable for measuring epitope spreading of antibody responses.

The assays of this invention are scored according to standard methods well known to those of skill in the art. The assays of this invention are typically scored as positive where there antibodies to one or more target epitopes (e.g. MOG conformational epitopes) are detected and/or quantified. In certain embodiments, the detection is with respect to one or more positive and/or negative controls. In certain embodiments, the “signal” is a detectable signal, more preferably a quantifiable signal (e.g. as compared to background and/or negative control).

It is noted that antibodies that bind to conformational epitopes of MOG are known to those of skill in the art (see, e.g., the Examples, herein, Sequences provided herein, and von Büdingen et al. (2002) Proc Natl Acad Sci USA, 99: 8207-8212). Proteins encoding such epitopes can readily be used in various assays (e.g. immunoassays) to detect and/or quantify anti-MOG antibodies, anti-Galc antibodies, and/or conformational eptope antibodies. Using such antibodies, and peptide/nucleic acid sequences for MOG (see, FIG. 31) provided herein, conformational epitopes can readily be identified and cloned using standard epitope mapping methods known to those of skill in the art. It is also noted that the foregoing assays and those illustrated herein in the Examples are intended to be illustrative and not limiting. Using the teaching provided herein numerous other asssays will be available to one of ordinary skill in the art.

Kits

In another embodiment, this invention provides kits for the screening procedures and/or diagnostic and/or prognostic procedures described herein. Screening/diagnositic kits typically comprise one or more reagents that specifically bind to the target that is to be screened (e.g. ligands that specifically bind to MOG conformational epitope antibodies). The reagents can, optionally, be provides with an attached label and/or affixed to a substrate (e.g. as a component of a protein array), and/or can be provided in solution. In certain embodiments, the kits comprise nucleic acid constructs (e.g. vectors) that encode one or more such ligands to facilitate recombinant expression of such. The kits can optionally include one or more buffers, detectable labels, or other reagents as may be useful in a particular assay.

In addition, the kits optionally include labeling and/or instructional materials providing directions (i.e., protocols) for the practice of the methods described herein. Thus, for example, in certain embodiments, preferred instructional materials describe the detection of MOG conformational epitope antibodies for the diagnosis, staging, and/or prognosis of multiple sclerosis and/or CIS.

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

### Molecular Characterization Of Antibody Specificities Against Myelin/Oligodendrocyte Glycoprotein In Autoimmune Demyelination

Multiple of the central nervous system (CNS) that is thought to be mediated by autoaggressive immune responses against myelin antigens (reviewed in Hohlfeld (1997) Brain 120: 865-916). Extensive investigations have addressed the respective roles of T and B cell responses against myelin antigens in experimental allergic encephalomyelitis (EAE), a disease model for MS. It is now recognized that, whereas myelin-reactive T cell responses are essential to disease pathogenesis, auto-Abs may play a major role as effectors of tissue damage (Hohlfeld (1997) Brain 120: 865-916; Bauer et al.(2001) Glia 36: 235-243; Brosnan and Raine (1996) Brain Pathol. 6: 243-257; Cross et al. (2001) J. Neuroimmunol. 112: 1-14). Myelin/oligodendrocyte glycoprotein (MOG) is a surface-exposed protein of myelin that has been identified as a prime target for demyelinating auto-Abs in several species (Genain et al. (1995) J. Clin. Invest. 96: 2966-2974; Linington et al. (1987) J. Immunol. 139: 4016-4021). Anti-MOG auto-Abs mediate a characteristic vesicular transformation of compact myelin in acutely demyelinating lesions, a neuropathological feature which has also been documented in human MS (Genain et al. (1999) Nat. Med. 5:170-175).

Despite these advances, the significance of polyclonal Ab responses against MOG measured in humans remains unclear. Anti-MOG Abs seem to be equally prevalent in the peripheral blood of affected patients and healthy controls (Karni et al. (1999) Arch. Neurol. 56: 311-315; Xiao et al. (1991) J. Neuroimmunol. 31: 91-96), and precise definition of the disease-relevant Ab epitopes of MOG is lacking. Similarly, the pathogenic significance of humoral responses directed against MOG has not been established with certainty for all EAE models (von Bu et al. (2001) J. Clin. Immunol. 21: 155-170). Indeed, these findings raise the possibility that the MOG-specific humoral response may be heterogeneous in terms of their potential to mediate demyelination. Analyses of the fine specificities of anti-MOG Abs in EAE and MS have mainly been conducted with short peptides derived from the amino acid sequence of MOG (Mesleh et al. (2002) Neurobiol. Dis. 9: 160-172; Haase et al. (2001) J. Neuroimmunol. 114: 220-225; Ichikawa et al. (1996) Int. Immunol. 8: 1667-1674). This approach cannot provide an understanding of the full complexity of anti-MOG humoral responses, because it does not account for epitopes that depend on the tertiary structure of the folded protein. Similarly, whereas molecular studies have independently established that CNS-specific clonal expansion of B cells occurs in MS (Qin et al. (1998) J. Clin. Invest. 102: 1045-1050; Owens et al. (1998) Ann. Neurol. 43: 236-243; Colombo et al. (2000) J. Immunol. 164: 2782-2789; Baranzini et al. (1999) J. Immunol. 163: 5133-5144), the antigenic specificities of these responses have not been identified. The use of systems that permit analysis of gene usage and individual Ab specificities should facilitate characterization of humoral responses against myelin autoantigens.

Here, we used a combinatorial Ab library of Fab fragments to characterize the humoral immune response against MOG in the common marmoset, an outbred primate species that develop an MS-like, Ab-mediated form of EAE after immunization with MOG (Genain and Hauser (1996) Methods 10: 420-434). We have observed that the recombinant MOG-specific Ab fragments use a limited repertoire of heavy (H)- and light (L)-chain genes and identify epitopes of MOG with specificities that are strictly conformation-dependent. The conformational epitopes of MOG defined by these Fab fragments are consistently targeted by the humoral repertoire in all outbred marmosets studied to date. Furthermore, we show that MOGimmune marmosets do not develop demyelinating EAE unless their humoral repertoire includes conformation-dependent Abs, a finding that underscores the relevance of this Ab subgroup in disease pathogenesis.

Materials and methods.

Animals and Induction of EAE.

All Callithrix jacchus marmosets used in this study were maintained in a primate colony at the University of California, San Francisco, and were cared for in accordance with all guidelines of the local Institutional Animal Care and Usage Committee. EAE was induced by active immunization with either 50 μg of recombinant protein corresponding to the extracellular domain of rat MOGaal-125 (rMOG) expressed in Escherichia coli and purified to homogeneity following published procedures (Amor et al. (1994) J. Immunol. 153: 4349-4356) or a mixture of 100 μg each of overlapping synthetic 20-mer peptides corresponding to the sequence of MOGaa1-120 (Research Genetics, Huntsville, Ala.). Peptides were purified >95% by HPLC, and purity was confirmed by mass spectrometry. Antigens were dissolved in 200 μl of PBS, emulsified with an equal volume of complete Freund's adjuvant, and injected intradermally as described (Genain et al. (1995) J. Clin. Invest. 96: 2966-2974). Animals were killed under deep phenobarbital anesthesia 4-70 days after the onset of clinical signs of EAE. Brain and spinal cord were dissected and fixed in 4% para-formaldehyde, and serial sections of the entire neuraxis were processed for routine histology.

TABLE 2
MOG-Fab IGHV and IGKV subgroup usage and H/κL-CDR3
motifs.

| | | | | | | | |
|---|---|---|---|---|---|---|---|
| | | | | | | % | |
| | | GenBank | | | GenBank | of | |
| | | Accession | | κL chain | Accession | total | Clone |
| IGHV | H chain CDR3 | No | IGKV | CDR3 | No | clones | ID |

| | | | | | | | |
|---|---|---|---|---|---|---|---|
| 1 | CARDVNFGNYFDY | AF393235 | 3 | CQQYSSWP | AF393236 | 73 | M26 |
| | (SEQ ID NO:1) | | | PTF | | | |
| | | | | (SEQ ID | | | |
| | | | | NO:2) | | | |
| 1 | CARDRGMGNYFDY | AF393237 | 3 | CQQYSSWP | AF393238 | 13 | M38 |
| | (SEQ ID NO:3) | | | LTF | | | |
| | | | | (SEQ ID | | | |
| | | | | NO:4) | | | |
| 1 | CARDATRILADVLDY | AF393239 | 3 | CQQYSSWY | AF393240 | 8 | M45 |
| | (SEQ ID NO:5) | | | TF | | | |
| | | | | (SEQ ID | | | |
| | | | | NO:6) | | | |
| 3 | CARAWRLSARAGY | AF393229 | 1 | CQQHYSTPL | AF393230 | 2 | M38 |
| | FDY | | | TF | | | |
| | (SEQ ID NO:7) | | | (SEQ ID | | | |
| | | | | NO:8) | | | |
| 3 | CILSDTGAFDV | AF393231 | 3 | CQQYSSWY | AF393232 | 2 | M324 |
| | (SEQ ID NO:9) | | | TF | | | |
| | | | | (SEQ ID | | | |
| | | | | NO: 10) | | | |
| 3 | CTGAGPTYYFDY | AF393233 | 1 | CQQGYTTP | AF393234 | 2 | M331 |
| | (SEQ ID NO:11) | | | VTF | | | |
| | | | | (SEQ ID | | | |
| | | | | NO:12) | | | |

Amino acid sequences of CDR3 motifs are deduced from cDNA. For complete H- and L-chain sequences please refer to GenBank.

Construction of a Combinatorial IgG-Fab Library from a MOGImmunized C. jacchus Marmoset.

The system used to generate the combinatorial library involved the phage display vector pCOMB3H (provided by C. F. Barbas III, The Scripps Research Institute, La Jolla, Calif.). This system permits the construction of a cloning product containing L and H chains flanked by SfiI restriction sites for directional cloning (Barbas et al. (2001) Phage Display: A Laboratory Manual (Cold Spring Harbor Lab. Press, Plainview, N.Y.)). Bone marrow and spleen cells were obtained from an rMOGimmunized C. jacchus that was killed after onset of clinical EAE. RNA was extracted with the Trizol reagent (Invitrogen) and first strand synthesis was performed with Superscript II reverse transcriptase (Invitrogen). In brief, three steps of PCR reactions were necessary to generate cloning inserts containing the Fab portions of C. jacchus IgG. (For a detailed description of these PCR steps, see Barbas et al. (2001) Phage Display: A Laboratory Manual (Cold Spring Harbor Lab. Press, Plainview, N.Y.)). First, all known marmoset H-chain variable region (IGHV) and SfilκL-chain variable region (IGKV) genes, as well as H-chain IgG CH1-domain and SfilκL chain C-region (IGKC) genes, were amplified in separate reactions (for primer sequences, see http\\itsa.ucsf.edu/claudeg/primers.htm and supporting information published on the PNAS web site, www.pnas.org). The template for IGHC and IGKC was a marmoset Fab library previously constructed in pCOMB3H, to include an SfiI restriction site on the 3′ end of IGHC and the pelB leader sequence with IGKC. In the second step, IGHV was joined with IGHC(H-chain assembly), and IGKV with IGKC (SfilκL-chain assembly). Third, the SfilκL chain (IGKV-IGKC-pelB) was joined with the H chain (IGHV-IGHC-SfiI) to yield a ˜1,460-bp cloning product containing SfiI-_L chain-pelB-H chain-SfiI. Finally, the cloning product and pCOMB3H were digested with SfiI (Roche Molecular Biochemicals) and purified. Equal amounts of pCOMB3H and C. jacchus VL/VH DNA were ligated with T4 ligase (Roche Molecular Biochemicals) and electroporated into electrocompetent XL1-Blue cells (Stratagene) with a Bio-Rad GenepulserII (2.5 kV, 200 ohms, 25 μF). The complexity of the obtained C. jacchus IgG_-pCOMB3H library was ˜1×10⁷ recombinants. Infective phagemid particles were generated by rescue with the helper phage VCSM13 (Stratagene).

Screening of the C. jacchus IgG-Fab Library with rMOG.

Approximately 1012 Fab-expressing phagemids were incubated (37° C., 1 h) in ELISA wells coated with rMOG (1 μg per well). In the first round of the selection process (panning), wells were washed 5 times with PBS containing 0.05% Tween20 (PBS-T), bound phagemid eluted with trypsin (500 μg per well), and eluted phagemid used to infect XL1-Blue cells. After incubation at 37° C. overnight, phagemids were precipitated and resuspended in PBS containing 1% BSA and submitted to the panning process 3 more times with increasing washing stringency (second round, 10 times; third round, 15 times; fourth round, 15 times). Enrichment of rMOG-specific Fab fragments was confirmed by measuring bound phagemid from each panning round in rMOG-coated ELISA wells with an anti-M13, horseradish peroxidaseconjugated Ab (Amersham Pharmacia Biotech).

DNA Sequence Analysis.

Phagemid DNA was extracted with the Qiagen (Valencia, Calif.) MaxiFilter kit and digested with SpeI and NheI for removal of the gIII protein gene, which permitted expression of soluble Fab fragments. SpeI_NheI-digested DNA was gel-purified, religated with T4 ligase, and transformed into XL1-Blue cells. Sixty randomly picked, Fab-expressing clones from the last panning round were grown in Superbroth containing 100 μg/ml of carbenicillin for minipreps, plasmid DNA was extracted with the Qiagen MiniPrep kit, and DNA was sequenced at the University of California, San Francisco, Genomics Core Facility by automated fluorescent chain termination sequencing. Sequences of both H- and L chains were aligned with MEGALIGN (DNAstar, Madison, Wis.).

Expression of Soluble Fab Fragments.

Fab-expressing clones representing all IGHV-IGKV combinations and H chain complementarity determining region (CDR) 3 motifs (Table 2) were grown in 3 liters of Superbroth until OD₆₀₀>1.2, and expression was induced with 1 mM IPTG. After overnight incubation at 30° C., bacteria were lysed by sonication in 30 ml of PBS and Fabs were purified from the soluble fraction over a protein L column (Pierce) following the manufacturer's protocol. Where desired, purified Fab fragments were biotinylated with a sulfo-Nhydroxysuccinimide (NHS) biotinylation reagent (Pierce) following the manufacturer's instruction. Unreacted sulfo-NHS biotin was removed by extensive dialysis against PBS.

Purification of Serum Anti-MOG Abs and Fractionation of Ab Specificities.

rMOG-reactive fractions of serum Abs were purified on 1-ml prepacked N-hydroxysuccinimide (NHS)-Sepharose columns reacted with 200 μg of rMOG, following the manufacturer's instructions (Amersham Pharmacia Biotech). rMOGSepharose columns were loaded with C. jacchus immune sera, diluted 1:5 in PBS, extensively washed with PBS, and bound Abs were eluted in 0.1 M glycine buffer, pH 2.2. For human sera, the protein G-reactive fraction (IgG) was extracted before purification by rMOG-affinity chromatography. To separate the Ab fractions binding to linear peptides from those binding to structural determinants of rMOG, MOG-peptide-reactive Ab fractions were removed from serum by repeated passes (n=5) on 1-ml PepMOG (mixture of overlapping 20-mer peptides spanning the entire sequence of rMOG) columns, which were synthesized by reacting 5.5 mg of PepMOG (500 μg per peptide) with NHS-Sepharose. Where desired, purified Abs were biotinylated as described previously.

Epitope Specificities of MOG-Reactive Ab and Recombinant Fab Fragments.

Maleic anhydride-activated ELISA plates (Pierce) were coated with 1 μg per well of rMOG or PepMOG (1 μg per peptide), blocked with PBS-T/3% BSA, and washed with PBS-T. Samples were added in PBS-T/3% BSA as follows: rMOG- or PepMOG-immune C. jacchus serum, or fractions thereof (after removal of peptide-specific Abs), 1:200; monoclonal Fab fragments, 1 μg per well. After incubation at 37° C. for 1 h, wells were washed with PBS-T, and appropriate secondary Ab added in PBS-T/3% BSA [serum and serum fractions: anti-monkey IgGhorseradish peroxidase (HRP) 1:6,000, Sigma; Fab fragments: protein L-HRP 1:5,000, Pierce] for 1 h at 37° C. After a final wash with PBS-T, plates were developed with tetramethylbenzidine (Pierce) and read at 450 nm.

Competition Assays.

Competition experiments were designed to examine the ability of Fab fragments to compete against each other and against native C. jacchus anti-MOG Abs for binding to rMOG. First, the amount of biotinylated Ab or Fab necessary to achieve 50% saturation of rMOG (50-100 ng per well) adsorbed on Ni-coated ELISA plates (Pierce) with biotinylated anti-MOG Abs or MOG-specific Fab was determined. To study competitive displacement, nonbiotinylated Fabs or native Abs were added to MOG-coated wells at increasing concentrations (10⁻¹² to 10⁻⁵ M) in the presence of the 50% saturation concentrations of the biotinylated reagent. After overnight incubation at 4° C., wells were washed and incubated with a streptavidin-peroxidase conjugate (Invitrogen; 1:1,000 in PBS-T/3% BSA, 20 min at room temperature). Bound Abs were detected with tetramethylbenzidine. Competition experiments with human anti-MOG Abs were performed with a similar protocol, in Immunosorp ELISA wells (Nunc) coated with rMOG (500 ng per well) in PBS. A constant concentration of unlabeled, MOG-affinity-purified Abs was incubated in the presence of increasing concentrations of the M26 and M3-8 Fab fragments overnight at 4° C. Bound human anti-MOG Abs were detected with an Fc-specific, alkaline phosphatase-conjugated anti-human IgG (Sigma, 1:5,000; this Ab was not cross-reactive with Fab fragments), and plates were developed with para-nitrophenol phosphate and read at 405 nm. Displacement was quantitated as the ratio of OD in the presence of competition over that in the absence of competition X 100 (%).

Immunohistochemistry.

Paraformaldehyde-fixed paraffinembedded sections of C. jacchus brain (7 μm) were deparaffinized, hydrated, and treated with a citrate-based antigenunmasking solution (Vector Laboratories) at high temperature for 20 min. Sections were blocked with 3% normal goat serum (Sigma) in PBS for 1 h at 37° C., washed with PBS-T, and incubated with biotinylated MOG-specific Fab (2.8 μg/ml) for 2 h at 37° C. Additional experiments were performed with the same dilutions of Fab fragments in the presence of rMOG to demonstrate specificity of binding. After incubation with the alkaline phosphatase (AP)-conjugated avidin complex [Vectastain ABC-AP (Vector Laboratories), 30 min, room temperature], fluorescence was revealed by the Vector Red AP substrate (Vector Laboratories), and slides were counterstained with hematoxylin.

Results

Ig Gene Usage of Recombinant MOG-Specific Fab Fragments.

Sixty randomly chosen, MOG-specific Fab-encoding clones were sequenced. The IGHV subgroup usage in this library was limited to IGHV1 and IGHV3, and IGKV usage to IGKV1 and IGKV3. Ninety-four percent (57 clones) of all clones were composed of IGHV1-IGKV3 (representative clones are designated M26, M38, and M45), and 6% were IGHV3-IGKV1 (M3-8, M3-31; 2 clones) or IGHV3-IGKV3 (M3-24; 1 clone). Sequences corresponding to contact residues (CDRs) showed considerable diversity, with variability in the H-CDR3 motifs (Table 2).

Recombinant Fab Fragments Exclusively Recognize Structural Epitopes of MOG.

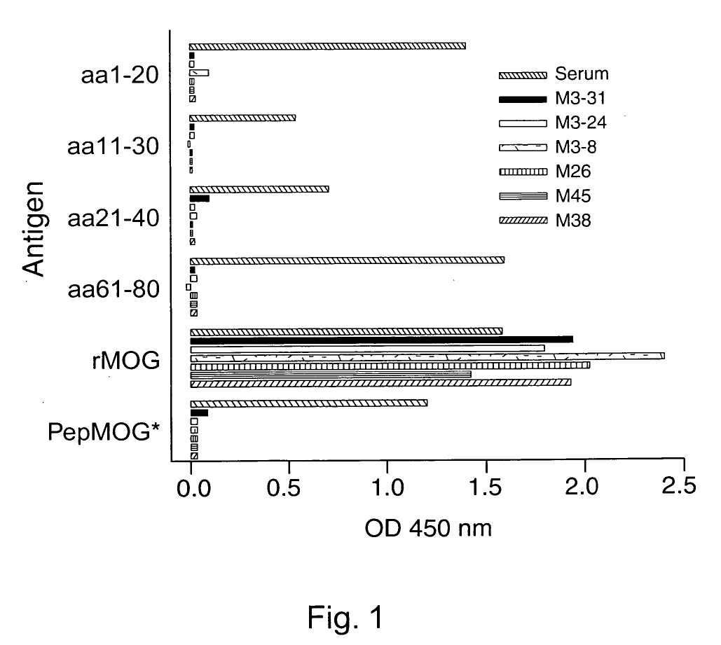

Polyclonal Ab populations present in serum of rMOG-immunized marmosets have been shown to recognize a broad repertoire of specificities, including linear epitopes corresponding to short peptide sequences contained within MOGaa1-125 (12, 22). Surprisingly, however, none of the recombinant Fab fragments studied showed binding to any of these linear, extended epitopes or to PepMOG (FIG. 1). Additional testing with an array of 60 overlapping 12-mer peptides confirmed these results (not shown). Thus, the MOG-specific Fab fragments selected from the combinatorial library exclusively recognized conformation-dependent epitopes.

Diversity of Structural Ab Epitopes of MOG.

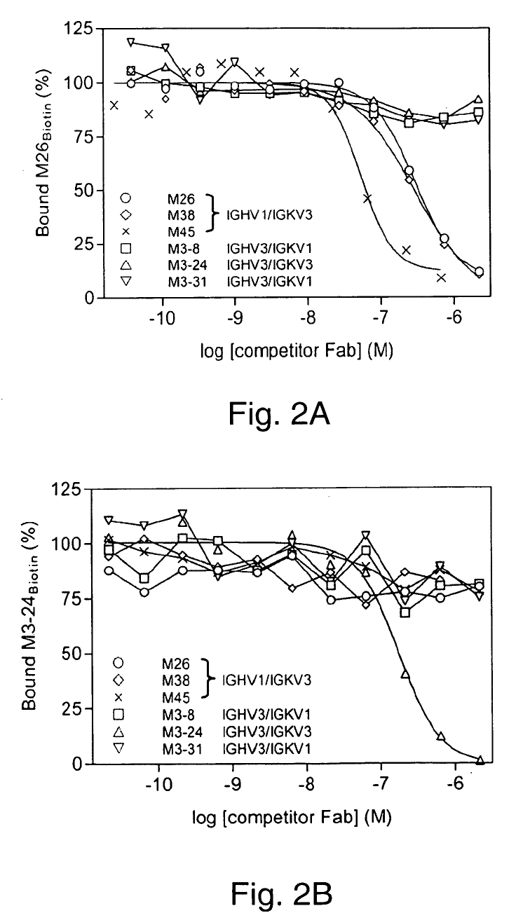

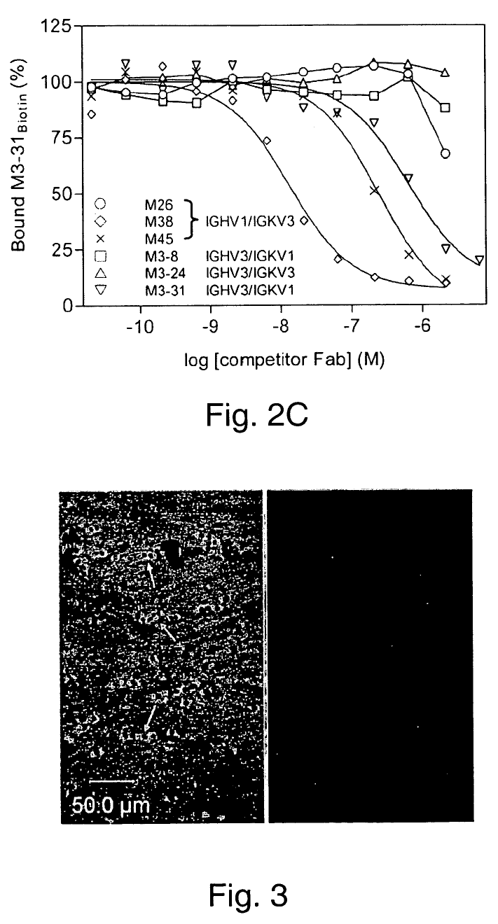

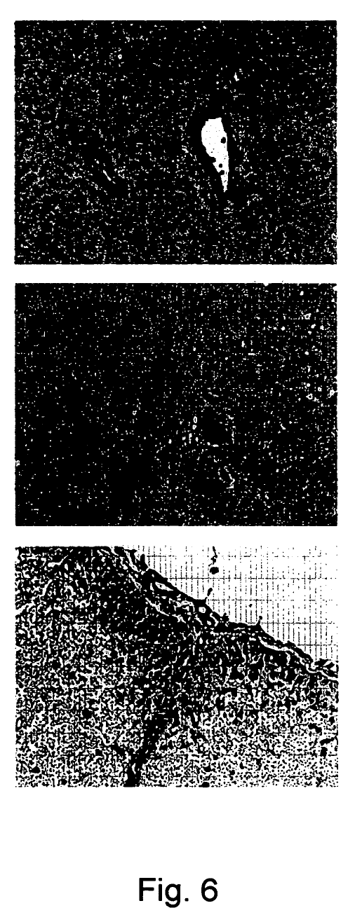

We performed competition experiments between Fab fragments representing all H-L chain combinations to understand the diversity of structural epitopes of rMOG targeted by the recombinant Fab fragments. Increasing concentrations of nonbiotinylated Fab fragments were allowed to compete in rMOG-coated ELISA wells with individual Fab fragments labeled with biotin. FIG. 2A illustrates the binding of a fixed amount of biotinylated M26 Fab (M26Biotin, IGHV1-IGKV3) in the presence of increasing concentrations of all other representative Fab fragments. Despite the variability in the CDR motifs (Table 2), all Fabs encoded by IGHV1-IGKV3 (M26, M38, and M45) recognize a similar epitope of rMOG. In contrast, no competition was observed between M26Biotin and M3-8, M3-31(IGHV3-IGKV1), or M3-24 (IGHV3-IGKV3). FIG. 2B illustrates a similar experiment with M3-24Biotin as the displaced Fab, which shows no competition with any of the other Fab fragments. These results indicate that the M3-24 Fab defines an epitope of rMOG that is distinct from that recognized by M26, M38, and M45. A similar lack of competition was observed for the M3-8Biotin Fab (not shown), suggesting that this IGHV3-IGKV1 combination defines another, unique conformational epitope. Subtle conformational features on exposed surfaces of MOG may be responsible for a microheterogeneity within the Abbinding sites. We found that M38 and M45 could displace the Fab M3-31Biotin, whereas only weak displacement by M26 occurred at significantly higher concentrations (FIG. 6C). Noncompetitive inhibition (e.g., steric interference) may play a role in this case and may explain the lack of displacement observed for the reverse experiment (e.g., M26Biotin vs. unlabeled M3-31, shown in FIG. 6A). Whether the M26 and M3-31 Fab fragments define similar or separate epitopes cannot be currently resolved. Nonetheless, these experiments identify at least three distinct conformational epitopes accessible on rMOG. All Fab fragments encoded by IGHV1_IGKV3 seem to recognize similar or closely associated epitopes on a single, major immunogenic region of MOG, which may be partially overlapped by M3-31.

Relevance of Antigenic Specificities Defined by Combinatorial Fab Fragments.

We next examined the ability of the recombinant Fab fragments to displace native anti-MOG Abs from C. jacchus serum, which represent a polyclonal mixture of Ab-specificities against linear determinants, structural determinants, or both. Biotinylated, affinity-purified anti-MOG Abs were incubated in the presence of increasing concentrations of Fabs. FIG. 2 shows representative experiments in which the M26 and M3-8 Fabs were allowed to compete against native, polyclonal auto-Abs from rMOG-immunized marmosets. Combinations of both M26 and M3-8 Fab fragments showed an additive effect for displacement, a finding that supports our hypothesis that the epitopes recognized by the M26 and M3-8 Fab fragments are topographically distinct (FIG. 3). Importantly, the representative Fabs derived from a single animal in this study efficiently displaced serum Abs from four genetically distinct marmosets (not shown). To verify that the Fab fragments were capable of binding to exposed epitopes of MOG on myelin sheaths, we confirmed by immunofluorescence that the recombinant Fab fragments were capable of binding to the MOG protein in situ in CNS white matter. FIG. 3 Left shows strong staining of oligodendrocytes and staining of myelinated fibers in C. jacchus corpus callosum with the biotinylated M26 Fab fragment. Specificity was confirmed by the ability to completely quench the fluorescent signal by addition of rMOG (FIG. 3 Right). Identical results were obtained with the M3-8 Fab fragment.

In Vivo Pathogenicity of Conformational Versus Linear Epitope-Specific Anti-MOG Abs.

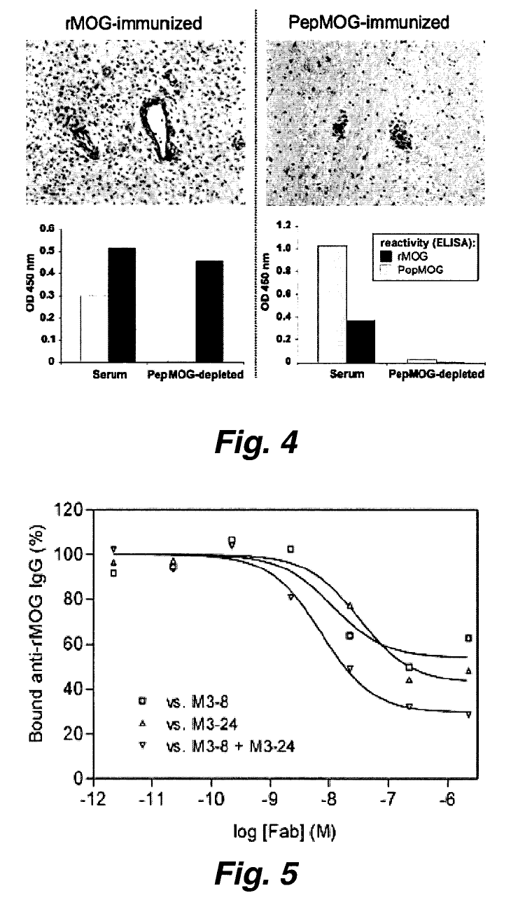

To understand further how epitope recognition influences Ab pathogenicity, we examined the binding characteristics of serum anti-MOG Abs in marmosets immunized with either rMOG (n=4) or PepMOG (n=2) before and after removal of the PepMOG-reactive fractions. Consistent with previous experience (Genain and Hauser (1996) Methods 10: 420-434), rMOG-immunized animals developed severe neurological signs corresponding to multifocal, widespread inflammatory infiltrates accompanied by prominent demyelination (FIG. 4). In contrast, animals immunized with PepMOG exhibited reduced disease burden with little or no demyelination. Importantly, we found that the repertoire of MOG-reactive Abs in this group was strictly restricted to linear epitopes, as removal of PepMOG-reactive Abs completely abolished reactivity to MOG. However, the sera from each of the rMOG-immune animals contained residual reactivity against whole rMOG after the complete removal of peptide-specific Abs (FIG. 4), indicative of the presence of immunogenic structural epitopes. Thus, the conformation-dependent Abs are only present in rMOG-immunized animals and seem to be responsible for the extensive demyelination observed in lesions of rMOG-induced EAE.

Marmoset Fab Fragments Delineate Structural Determinants of the MOG Ab Response in Humans.

We examined the ability of recombinant marmoset Fab fragments to displace affinity-purified anti-MOG Abs from the sera of three patients with MS (AA, DM, and WS), who were previously shown to be MOG-reactive by ELISA. We found that the M3-8 fragment was able to compete with anti-MOG Abs from all three patients and also found competition with M3-24 for patient AA. Furthermore, similar to marmosets, the combination of M3-8 and M3-24 showed an additive effect (FIG. 5, representative experiment; patient AA). These results indicate that the targets for MOGspecific Abs in humans include conformation-dependent epitopes that are identical to those in marmosets.

Discussion

In this report we provide information regarding the molecular complexity of pathogenic auto-Ab responses against exposed domains of MOG in an outbred species.

Previous studies reporting the effects of passive transfer of certain Abs in rodent and marmoset systems (Genain et al. (1995) J. Clin. Invest. 96: 2966-2974; Brehm et al. (1999) J. Neuroimmunol. 97: 9-15), and of MOG-DNA vaccination in SJL mice (Bourquin et al. (2000) Eur. J. Immunol. 30: 3663-3671), have shown that conformation-dependent anti-MOG Abs are capable of inducing demyelination. In contrast, whether Abs directed at linear determinants of MOG have demyelinating properties has not been unequivocally demonstrated (Ichikawa et al. (1996) Int. Immunol. 8: 1667-1674; Adelmann et al. (1995) J. Neuroimmunol. 63: 17-27). Data obtained from two PepMOG-immunized animals in this study suggest that the presence of MOG-peptide-specific Abs is not associated with widespread demyelination. Indeed, EAE in these animals was reminiscent of the disease phenotype produced by adoptive transfer of MOG-reactive T cells (Villoslada et al. (2001) Eur. J. Immunol. 31: 2942-2950). Similar EAE phenotypes could also be reproduced in animals immunized with groups of individual peptides that contain the marmoset immunodominant MOG-T cell epitopes (n=7) (von Budingen et al. (2001) J. Clin. Immunol. 21: 155-170). Taken together, these results suggest that Abs against linear peptides are not pathogenic in marmosets and that recognition of conformational features of MOG is a prerequisite for Ab pathogenicity.

Peptide-specific anti-MOG Abs are part of the MOG-immune repertoire in EAE and can be detected in the serum of healthy controls and patients with MS (. Karni et al. (1999) Arch. Neurol. 56: 311-315; Xiao et al. (1991) J. Neuroimmunol. 31: 91-96). However, because of the stringent conditions applied during the panning process, it is likely that the conformational epitopes of rMOG define binding sites for Abs of higher affinity than MOG-peptide Abs, which were not found in the Fab library. Similar differences in affinity have been described in the case of a different antigen (Sachs et al. (1972) Proc. Natl. Acad. Sci. USA 69: 3790-3794; Jemmerson and Blankenfeld (1989) Mol. Immunol. 26: 301-307). Nonetheless, we show that the Fab fragments specifically bound to native MOG in situ in brain tissue, indicating that our combinatorial approach had yielded Ab fragments that correctly define structural features of MOG that are exposed in vivo. The relevance of these Ab fragments is underlined further by our finding that the monospecific Fab fragments are capable of displacing a significant portion of the polyclonal, native anti-MOG Abs in several marmosets, despite the genetic heterogeneity present between outbred individuals. Thus, despite the fact that our library may not exhaustively include all Ab specificities present in the polyclonal, MOG-specific humoral repertoire, we propose that the MOG-specific Fab fragments represent epitope specificities with demyelinating potential.

Accurate definition of the determinants of MOG that are targets of demyelinating Abs in humans will be of critical importance. Qualitative differences in epitope recognition may be present among anti-MOG Ab populations that are frequently detected in patients with MS and healthy controls (Haase et al. (2001) J. Neuroimmunol. 114: 220-225; Reindl et al. (1999) Brain 122: 2047-2056; Sun et al. (1991) J. Immunol. 146: 1490-1495). For example, T cell mimicry between viral antigens and MOG peptides has been reported (Mokhtarian et al. (1999) J. Neuroimmunol. 95: 43-54), but in the absence of exposure of B cells to the whole MOG polypeptide, may only induce production of MOGpeptide-specific Abs. These auto-Abs would be detected in standard Ab assays, although they may not be pathogenic. We show here that Ab fragments that define structural determinants of MOG in C. jacchus can be used to specifically detect the presence of MOG-specific idiotypes directed against identical determinants in humans. Although in marmosets the M26 and M3-8 Fab fragments seem to represent important specificities, in serum from MS patients the M3-8 and M3-24 Fabs have thus far been shown to compete against native anti-MOG Abs. Additional experiments are proceeding to identify human Abs capable of competing with the remaining marmoset Fabs.

Clonal B cell expansion with restricted usage of IGHV germline genes in CNS lesions and cerebrospinal fluid of patients with MS has been reported (Qin et al. (1998) J. Clin. Invest. 102: 1045-1050; Owens et al. (1998) Ann. Neurol. 43: 236-243; Colombo et al. (2000) J. Immunol. 164: 2782-2789; Baranzini et al. (1999) J. Immunol. 163, 5133-5144). In this context, it was of interest to find that a limited number of H- (IGHV1 and IGHV3) and _L-chain (IGKV1 and IGKV3) subgroup genes was used in the marmoset MOG-specific Ab repertoire. However, we also found that diverse CDR-encoding gene rearrangements were used to target only three epitopes of MOG. Therefore, the current study extends beyond prior molecular analyses of Ig gene usage, which have not identified target antigens for the clonally expanded immune responses. Competition experiments also demonstrated that the C. jacchus Fab fragments define antigenic determinants of MOG that are commonly targeted in all marmosets, regardless of H- and L-chain usage.

The critical importance of MOG to autoimmune demyelination is a consequence of its restricted expression in the CNS (Gardinier et al. (1992) J. Neurosci. Res. 33: 177-187), its exposed extracellular domain at the outermost lamellae (Brunner et al. (1989) J. Neurochem. 52: 296-304), its high level of encephalitogenicity in multiple species, and tendency to induce pathogenic auto-Ab responses directed against the myelin sheath (reviewed by von Bu et al. (2001) J. Clin. Immunol. 21: 155-170). The finding reported here that related conformational features of MOG are targets of auto-Abs in marmosets and humans highlights the value of nonhuman primate models for dissection of auto-Ab responses relevant to the pathophysiology of CNS tissue damage in MS.

### Example 2

### The Use of Epitope Specific Antibodies For Diagnosis and Prognosis of Multiple Sclerosis

Results.

A) Complexity of Autoantibody Responses Against MOG in Outbred Species

1. Repertoire Heterogeneity.

In marmosets immunized with the extracellular domain of MOG (aa₁₋₁₂₅, rMOG), mapping of rMOG-specific antibody specificity in sera and CSF using short peptides revealed 2 immunodominant regions, MOG aa₁₃₋₂₁ (100% of animals) and MOGaa₆₃₋₇₅ (85%). Additional reactive peptides were identified at residues aa₂₈₋₃₅ and aa₄₀₋₄₅. Some of the B cell epitopes in marmosets match the location of T cell epitopes (Brok et al. (2000) J. Immunology, 165(2):1093-1101; von Büdingen et al. (2001) J. Clin. Immunol., 21(3):155-170; Mesleh et al. (2002) Neurobiol Dis., 9(2):160-172), as has been shown for MOG in rodents (Ichikawa et al. (1996) J. Immunol., 157:919-926), and for an immunodominant epitope of MBP in humans (Wucherpfennig et al. (1997) J. Clin. Inves., 997: 100(5): 1114-1122). These linearly defined epitopes include aa residues that are located on accessible regions of the molecule, according to predictive models for the structure of MOG (Mesleh et al. (2002) Neurobiol Dis., 9(2): 160-172). Epitope diversity in the antibody repertoires against MOG in outbred species is confirmed by studies of MOG reactivity in macaque monkeys (de Rosbo et al. (2000) J. Neuroimmunol. 110:83-96), and humans (de Rosbo et al. (1997) Eur. J. Immunol., 27(11):3059-69; Lindert et al. (1999) Brain 122(Pt11):2089-2100).

2. Functional Heterogeneity Within Anti-MOG Antibodies.

Pathogenic properties of autoantibodies can be directly demonstrated in experimental systems that use combinations of adoptive transfer of T cells, and passive transfer of autoantibodies with demyelinating properties (Genain et al. (1995) J. Clin. Invest. 96: 2966-2974; Lassmann et al. (1988) Acta Neuropathol. (Berl) 75: 566-576; Schluesener et al.(1987) J Immunol 139(12):4016-4021). C. jacchus marmosets do not develop severe EAE associated with prominent demyelination after immunization with MBP, or adoptive transfer of MBP- or MOG-specific T cell clones (Genain et al. (1994) J. Clin. Invest. 94: 1339-1345; Villoslada et al. (2001) Eur. J. Immunol. 31: 2942-2950), in contrast to immunization with whole white matter, or with rMOGaa 1-125. Non-demyelinating EAE can be converted to fully demyelinating disease by passive transfer of rMOG-, or whole white matter-reactive IgG, indicating that these preparations contain pathogenic autoantibodies.

To investigate whether pathogenicity is dependent on epitope recognition, we have studied the clinical and pathological phenotypes of EAE induced in C. jacchus with 20 mer linear peptides encompassing the most frequently recognized epitopes within human rMOG. Active immunizations with 100 μg of MOG aa₂₁₋₄₀, combinations of equal amounts of peptides spanning the reactive T cell epitopes (aa₂₀₋₄₀, aa₆₃₋₇₂, aa₉₁₋₁₁₀), or combination of each of 11 overlapping peptides corresponding to the entire sequence of human rMOG, reproducibly induced mild, chronic EAE (n=2 per group), although a severe clinical course associated with a large solitary cervical cord lesion was observed in one animal immunized with a mixture of all the overlapping peptides. In all other animals, the mild clinical phenotype correlated with small inflammatory infiltrates, accompanied by sparse demyelination (FIG. 6).

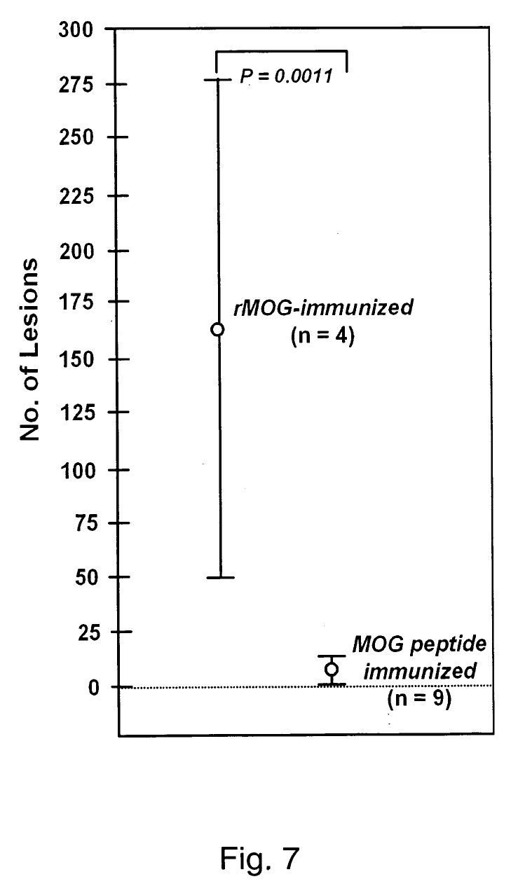

Reminiscent of adoptive transfer EAE in this species (Villoslada et al. (2001) Eur. J. Immunol. 31: 2942-2950), pathology remained scarce (<10 infiltrates within the entire neuraxis, FIGS. 6 and 7) and mostly confined to the cervical spinal cord. Despite the production of robust T cell responses against the immunizing peptides that paralleled those measured in rMOG-immune animals, no combination of peptides was capable of reproducing the protracted, multifocal disease associated with prominent demyelination that typically results from immunization with whole rMOG in this species.

3. Fractionation of polyclonal MOG-reactive autoantibody populations.

MOG peptide- and rMOG-reactive antibodies were separated by affinity chromatography on Sepharose columns containing MOG peptides covalently bound to Sepharose. In rMOG-immune marmosets, serum antibodies appeared to contain one fraction that recognized both linear MOG peptides and the whole rMOG polypeptide, and a second fraction that exclusively recognized conformational determinants (FIG. 8A, red bars). ELISA of the bound material after elution demonstrated that this second fraction contained antibodies that are capable of binding to rMOG, in addition to MOG peptides (FIG. 8-A, blue bars). By contrast, all sera from animals immunized with 20 mer overlapping peptides of rMOG (individually or in combination, n=9), only contained antibodies binding to the immunizing peptides. The MOG peptide-specific antibodies present in these animals did recognize rMOG (FIG. 8B, green bars), but not its conformational determinants, as shown by removal of all reactivity after depletion on the MOG peptide columns. (FIG. 8B, red bars).

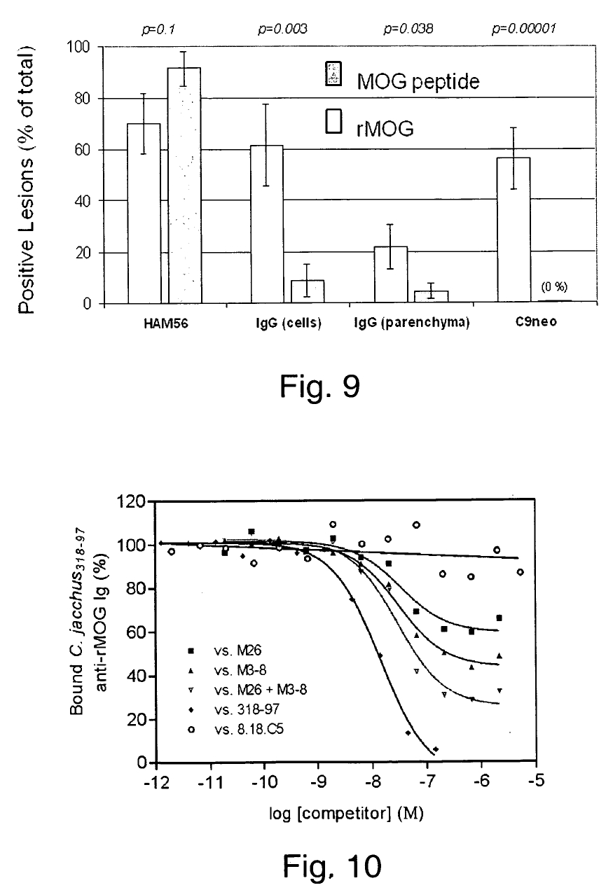

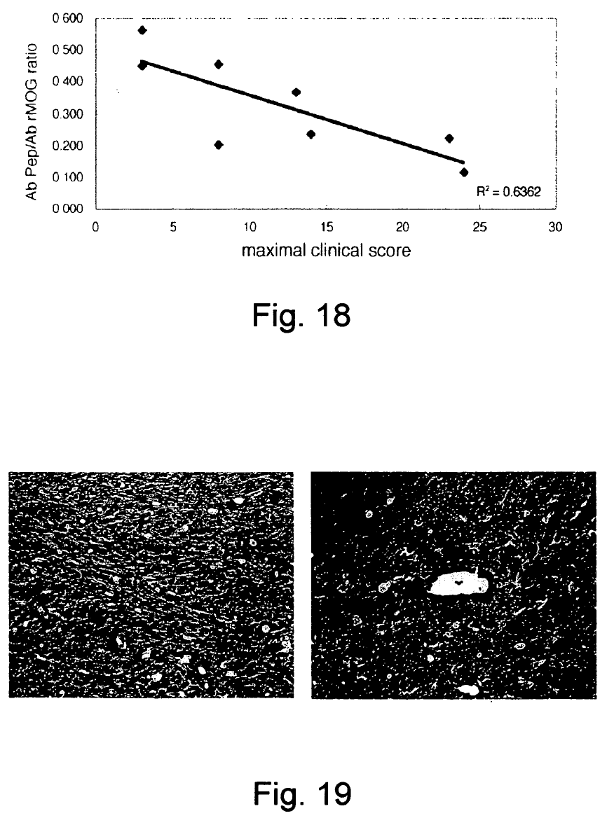

These data demonstrate that MOG-reactive autoantibodies in marmosets are heterogeneous in terms of epitope recognition, and may be directed against 3 different classes according to their binding characteristics to conformational rMOG, linear rMOG-derived peptides, or both. As illustrated in FIG. 8, it is not possible to distinguish these different antibody fractions by ELISA or other standard antibody detection methods using whole serum. The difference in epitope recognition appears to translate into functional heterogeneity (e.g., pathogenic potential). Immunohistochemical analysis in marmosets immunized with the linear peptides showed lesion patterns that were strikingly different from those in rMOG-immune animals. Whereas macrophage infiltration was equally present in both forms of EAE, Ig and complement deposition were uniformly absent in the peptide-immune animals (FIG. 9). We have also observed that disease severity in rMOG-induced marmoset EAE is inversely proportional to the ratio of serum concentrations (μg/ml) of MOG peptide/rMOG-reactive IgG (Tanuma et al. (2001) J. Neuroimmunol., 118:60, and FIG. 18).

These findings are in agreement with studies of the properties of murine monoclonal antibodies (Brehm et al. (1999) J. Neuroimmunol., 97:9-15), and studies of EAE in C57/B16 wild type and B cell KO mice immunized with either whole rMOG or MOG peptide aa₃₅₋₅₅ which is immunodominant in rodents (Lyons et al. (1999) Eur. J. Immunol., 29(11):3432-3439; Svensson et al. (2002) Eur J. Immunol., 32(7): 1939-46; Lyonset al. (2002) Eur J. Immunol., 32(7): 1905-1913; Albouz-Abo et al. (1997) Eur J. Biochem., 246(1):59-70). It is noteworthy however, that severe demyelinating EAE can clearly be induced by active immunization with linear peptides of MOG in several rodents strains (Bernard et al. (1997) J. Mol. Med., 75(2):77-88; Slavin et al. (1998) Autoimmunity, 28(2):109-120; Amor et al. (1994) J. Immunol., 153:4349-4356; Tsunoda et al. (2000) Brain Pathology, 10(3):402-418; Ichikawa et al. (1996) International Immunology, 8(11): 1167-1674). The apparent discrepancy may reflect differences in strain/species susceptibility, and supports an hypothesis that genetic background may selectively determine factors of disease pathogenesis that translate into variation in the clinical and pathological phenotype of CNS demyelinating disease. Our combined observations that MOG peptide-specific antibodies do not appear to be demyelinating in marmosets, but can be detected in situ in active lesions of EAE and MS (Genain et al. (1999) Nature Medicine, 5:170-175), also suggest that certain autoantibodies are cross-reactive to both linear and conformational determinants. As will be discussed below, similar heterogeneity exists within anti-MOG humoral responses in humans. Pathogenicity of the different classes of antibodies to MOG has not been clearly defined.

Without being bound by a particular theory, we believe that autoantibodies restricted to selective determinants of MOG cause demyelination in human MS, and influence clinical course. The complex patterns of serum antibody responses are correlated with disease phenotype and their pathogenic potential.

B) Molecular and Structural Complexity of MOG-Specific Antibody Repertoires in Outbred Species.

The molecular diversity of MOG-specific C. jacchus antibody repertoires was analyzed using phage-displayed combinatorial libraries of Fab fragments from bone marrow and spleen obtained from animals immunized with rat rMOG (after they had developed antibody responses and clinical EAE), using sequence information previously obtained for the variable regions of H chains (V_{H}) and the k L chains (Vk) (von Büdingen et al. (2001) Immunogenetics, 53:557-563; von Büdingen et al. (2002) Proc. Natl. Acad. Sci. USA, 99(12):). Sequence analysis of clones from the first library showed predominant usage of C. jacchus V_{H}1 and VkIII rearrangements (94% of all combinations), while VH3/VkI and VH3/VkIII represented the remaining combinations. Six H-CDR3 motifs and 5 different kL-CDR3 were identified, with the greatest degree of variability in the H-CDR3 motifs. A second rMOG-immune library has been constructed from a genetically distinct marmoset, and is currently been analyzed. Data from 30 clones of this library appear to confirm the predominant usage of V_{H}1, V_{H}3 and VkIII. Soluble, recombinant Fab fragments were expressed from selected Fab-producing clones, purified on protein L-affinity columns, and analyzed for their binding properties by ELISA. In contrast to whole serum or antibody fractions from immune C. jacchus, all Fab fragments representative for the V_{H}/Vk rearrangements failed to show binding to any of 20 mer overlapping linear peptides spanning the sequence of rMOG (FIG. 1), or to a panel of 96 overlapping peptides corresponding to the sequence of MOG aa1-120.

The availability of these monoclonal, recombinant Fab fragments from C. jacchus afforded to design competition experiments, where displacement of one biotinylated Fab fragment by other Fabs is measured. This technique affords to study the diversity of epitopes available for antibody binding on rMOG at the structural level. All Fab fragments encoded by V_{H}1/VkIII rearrangements were shown to fully compete with each other, even though these clones utilized different H-CDR3 motifs. The M3-8 and M3-24 Fab fragments only competed against themselves, while the M3-31 was shown to compete against both M38 and M45. M26 only weakly displaced M3-31 at high concentrations, suggesting partial overlap between the epitopes defined by those 2 Fab fragments. These data suggest that the diversity of humoral responses to MOG observed at the nucleic acid sequence level does not translate into the same degree of diversity for structural antigenic specificities. Three different conformational antibody epitopes have been identified so far, suggesting the existence of a limited number of accessible binding sites on the intact rMOG polypeptide. Work is in progress to similarly characterize additional MOG-immune Fab libraries and will provide a complete definition of anti-MOG antibody repertoire in C. jacchus.

C) Relevance of Combinatorial Studies to Native Autoantibody Repertoires

The biological relevance of the randomly rearranged recombinant Fab fragments was tested by measuring their ability to compete with biotinylated rMOG-specific IgG fractions purified by affinity chromatography from serum of marmosets with rMOG EAE. Fab fragments M26 and M3-8 efficiently displaced these polyclonal antibodies, indicating that the combinatorial approach produced antibody specificities that reflected the mature, rMOG-driven IgG repertoire (FIG. 10). Similar conclusions have been derived from other studies using combinatorial antibody technology (Barbas et al. (2001) Phage Display. A laboratory manual. Cold Spring Harbor, N.Y.: Cold Spring Harbor Laboratory Press). Importantly, displacement of native, rMOG-immune serum IgG could be demonstrated for 4 unrelated animals, indicating that the epitope specificities captured by the combinatorial approach correspond to specificities that are commonly targeted in marmosets, despite their outbred genetic characteristics.

In contrast to C. jacchus recombinant Fabs, the murine monoclonal antibody 8.18.C5 was not capable of displacing MOG-immune C. jacchus IgG. We also observed that none of the recombinant C. jacchus Fab fragments studied to date, were capable of competing for binding with the 8.18.C5 antibody. Although this antibody recognizes rat, mouse, human and marmoset MOG, and is capable of inducing demyelination in C. jacchus (Genain et al. (1995) J. Clin. Invest. 96: 2966-2974) and other species (Liningtonet al. (1988) Am J Pathol., 130(3):443-454; Schluesener et al.(1987) J Immunol 139(12):4016-4021), our results indicate that the epitope defined by 8.18.C5 is not part of the natural C. jacchus repertoire against MOG.

D) Relevance of C. jacchus Studies to Pathogenic Antibody Responses in Humans

1. Competition between C. jacchus Fab fragments and MS serum antibodies.

Competition experiments were extended to Ig (IgG and IgM) present in the serum of patients with MS. rMOG-specific Igs were affinity purified on MOG-Sepharose columns, and tested for their ability to displace recombinant C. jacchus Fab fragments from rMOG in ELISA wells. To date, we have identified 3 patients in a group of 6 who displayed Ig reactivity to rMOGaa1-125 containing conformation-dependent antibodies that can be displaced by the C. jacchus Fab M3-8, and/or the M3-24 Fab (FIG. 5). A fourth patient exhibited antibodies against linear peptides, but not conformational determinants, of MOG (please also refer to Table 3, below).

These experiments underscore the value of studies of C. jacchus antibody repertoires as the first practical tools to define the target epitopes of MOG for pathogenic antibody responses in MS, and demonstrate that certain conformation-dependent epitopes of MOG are unique to primate species. The structural diversity of human anti-MOG antibodies readily be further assessed by approaches similar to those described for C. jacchus.

2. C. jacchus Fab fragments recognize human MOG in situ.

C. jacchus Fab fragments were tested for their ability to bind to MOG under conditions that mimic exposed epitopes of the MOG molecule in vivo. First, we confirmed that these fragments could bind to CNS myelin sheaths and oligodendrocytes by Immunohistochemistry in sections of C. jacchus and CNS brain (von Büdingen et al. (2002) Proc. Natl. Acad.Sci. USA, 99(12)). Second, we generated several cell lines transfected with a plasmid encoding for the full-length sequence of human MOG (aa₁₋₂₁₈), including COS cells, a human fibroblast cell line, and the human oligodendroglioma cell line TC 620. All C. jacchus Fabs tested to date are capable of binding to transfected, but not to untransfected cells.

3. Demyelinating potential of MOG-specific autoantibodies present in humans.

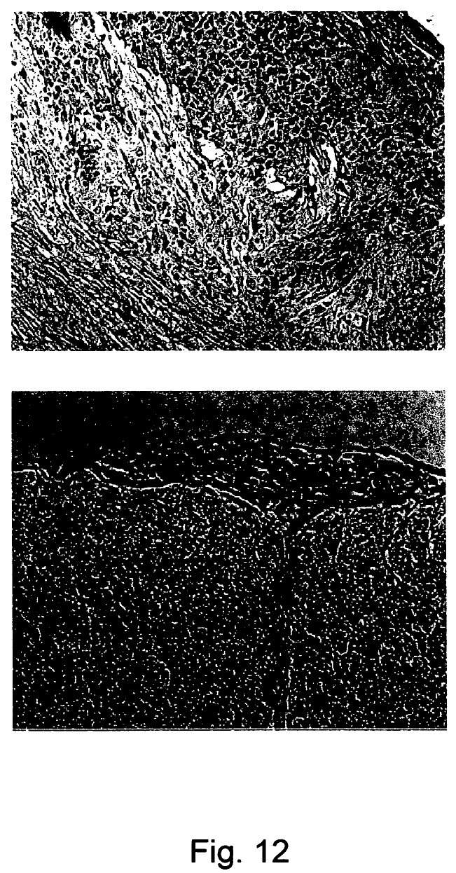

In preliminary experiments, we have performed passive antibody transfers in 2 MBP-immunized marmosets with IgG purified from patients with MS that tested positive for anti-MOG antibodies by ELISA. Similar to homologous IgG transfers, administration of these human antibody fractions in C. jacchus readily induced large EAE lesions with prominent demyelination (FIG. 12). These experiments indicate that the C. jacchus passive antibody transfer system is suitable to to assess pathogenicity of human anti-MOG antibodies.

E) Reactivity to Myelin Antigens in MS

Specific ELISA systems have been developed in the laboratory to assess reactivity of human sera to MBP, rMOG, MOG-derived peptides and Galc (IgG and IgM).

1, rMOG-, MOG peptide- and MBP-reactive autoantibodies.

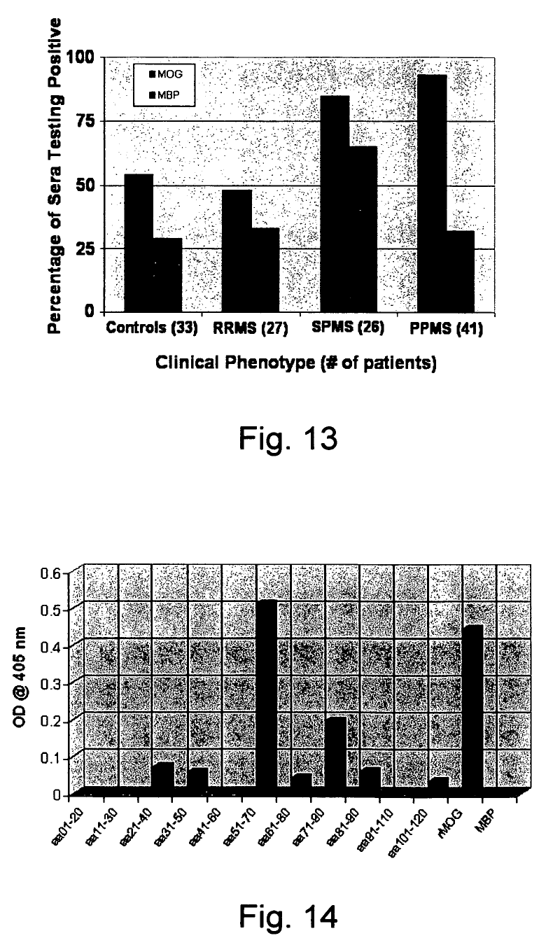

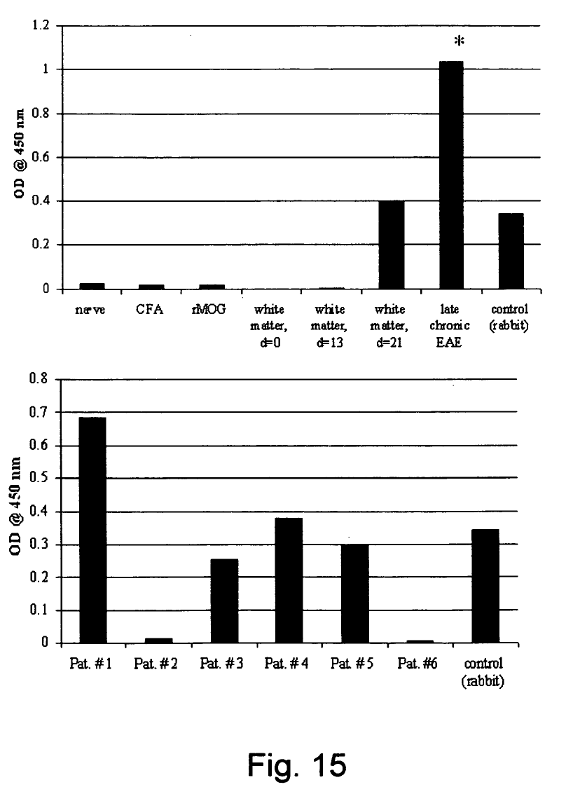

We have examined a series of 33 age-matched controls (including older subjects to better match the age distribution of progressive MS), 27 patients with relapsing remitting (RR) MS, 26 with secondary progressive (SP) MS, and 41 with primary progressive (PP) MS, for serum reactivity to rMOG and MOG-derived peptides. While the frequency of anti-MOG antibodies is sensitively higher in the controls (54%) but still consistent with previous work by others (Reindl et al. (1999) Brain, 122:2047-2056; Lindert et al. (1999) Brain 122(Pt11):2089-2100; Egg et al. (2001) Mult Scler., 7(5):285-289), the data confirm the high prevalence of anti-MOG antibodies in the SPMS and PPMS (85% and 93% respectively) and the high prevalence of anti-MBP antibodies in SPMS (FIG. 13).

Similar to studies of C. jacchus and macaque monkeys (Mesleh et al. (2002) Neurobiol Dis., 9(2): 160-172;de Rosbo et al. (2000) J Neuroimmunol., 110(1-2):83-96), linear epitopes recognized by human anti-MOG antibodies showed great diversity and variability between individual patients. In a total of 16 rMOG-reactive patients studied for fine mapping with overlapping 20 mer peptides (10 relapsing remitting and 6 secondary progressive), the most frequently recognized motifs included aa₂₁₋₄₀, aa₃₁₋₅₀, aa₅₁₋₇₀, aa₇₁₋₉₀, and aa₁₀₁₋₁₂₀. We also observed a significant proportion of patients whose serum did not react with whole rMOG, but reacted to linear peptides. Analysis of serum reactivity using an extensive panel of 96 overlapping peptides corresponding to MOGaa₁₋₁₂₀, and portions of the C-terminus of human MOG, confirmed this diversity of linear epitopes. No distinctive pattern of epitope reactivity in relation to clinical phenotype was apparent in this small sample. These results are in agreement with previous studies of antibody reactivity to MOG in MS (de Rosbo et al. (1997) Eur. J. Immunol., 27(11):3059-3069; Haase et al. (2001) J Neuroimmunol., 114(1-2):220-225), and confirm diversity in human anti-MOG antibody repertoires.

2. Detection of conformation-dependent MOG autoantibodies in human MS.

The presence of antibodies that exclusively recognize conformational determinants of MOG within complex polyclonal antibody responses is difficult to assess using standard detection techniques. These antibodies can be physically separated from those recognizing linear determinants. However, the technique is not suitable to distinguish between antibodies that are strictly conformational, and those that may recognize both conformational determinants and linear peptides, because there likely are antibodies that bind to both linear peptides and rMOG (this Section, FIG. 8). We have taken advantage of specific competition assays using biotinylated recombinant, monoclonal C. jacchus Fab fragments that exclusively define conformational epitopes to investigate the presence of conformation-dependent antibodies within the repertoire of patients that tested positive for anti-rMOG antibodies. To date, we have examined in detail the fine specificities of anti-MOG antibodies (IgG and IgM) in 6 individuals with RRMS or SPMS. Data presented in Table 3 summarize these findings, and also show that IgG and IgM antibodies can be independently involved in the MOG-specific human humoral response.

TABLE 3
Reactivity of MS sera to rMOG, MOG derived peptides, and
competition experiments with the conformation-dependent
C. jacchus Fabs. PepMOG designates one or more reactive
peptides within MOGaa₁₋₁₂₀.

| | | | | | Competition with | | |
|---|---|---|---|---|---|---|---|
| Sub- | IgG (ELISA) | | IgM (ELISA) | | C. jacchus Fab | | |
| ject | rMOG | PepMOG | rMOG | PepMOG | M3-8 | M26 | M3-24 |
| 1 | + | + | + | + | Yes | No | ND |
| 2 | − | + | ND | ND | No | No | No |
| 3 | + | + | + | + | Yes | No | Yes |
| 4 | + | + | + | + | Yes | No | ND |
| 5 | − | − | − | − | No | No | No |
| 6 | + | + | − | − | No | No | ND |

ND, not done.

Based on these findings, it is possible to envision that responses to MOG in humans segregates into subtypes depending on the pattern of epitope recognition. Of interest is the relative pathogenic potentials of these different human antibody populations. Heightened incidence of anti-MOG antibodies in our cohorts of SPMS and PPMS patients suggest that these antibodies are consistently associated with late forms, or progressive forms of disease typically characterized by severe disability and CNS atrophy.

3. Reactivity to MOG in patients with clinically isolated syndromes

We have studied the sera of 8 patients that presented with a clinically isolated syndrome (CIS) associated with MRI abnormalities (Table 4). Six of these subjects displayed strong serum reactivity to MOG and/or MOG peptides. These data indicate that anti-MOG antibodies are a prominent part of the immune response during the first detectable clinical event in MS, in agreement with previous studies of early relapsing remitting MS (Reindl et al. (1999) Brain, 122:2047-2056).

An ongoing collaboration between the neuroimmunology laboratory and the UCSF MS and MRS Centers has begun to analyze correlations between serologic measurements (anti-myelin antibodies), and clinical and MRI phenotypes. Results for the first 11 CIS patients studies are shown below (Table 4).

TABLE 4
Clinical, MRI, and serologic characteristics of CIS patients.

| | | | | T2 | | | | | |
|---|---|---|---|---|---|---|---|---|---|
| | | Resolved | | Lesion | | # of | | | |
| | Clinical | at | EDSS at | Load | # of T2 | Gd+ | α- | α- | α-MOG |
| ID | Presentation | sampling | sampling | (cm³) | Lesions | Lesions | MBP | rMOG | peptides |
| 1 | Myelitis | improved | 2.0 | 2.077 | 16 | 0 | − | + | − |
| 2 | Myelitis | improved | 1.5 | 0.117 | 3 | 0 | − | + | − |
| 3 | Brainstem | improved | 1.5 | 0.035 | 2 | 0 | − | + | + |
| 4 | Brain Stem | No | 1 | 2.432 | 16 | 0 | + | − | − |
| 5 | Myelitis | No | 3 | 0.656 | 13 | 4 | − | + | + |
| 6 | Optic | improved | 1.5 | 0.622 | 9 | 0 | − | − | + |
| | neuritis | | | | | | | | |
| 7 | Myelitis | Yes | 0 | 0.387 | 5 | 1 | + | + | ND |
| 8 | Myelitis | improved | 1.5 | 0.252 | 5 | 1 | − | − | ND |
| 9 | Myelitis | | 2.0 | 3.811 | 19 | 0 | − | + | − |
| 10 | Myelitis | | 0 | 4.534 | >40 | 0 | − | − | − |
| 11 | Brain Stem | | 0 | 1.807 | 6 | 0 | − | + | − |

ND: not done.

These data, although limited to a small number of patients that does not permit statistical analysis, very clearly indicate that anti-MOG antibodies are a prominent part of the immune response during the first detectable clinical event in MS. This finding is in agreement with previous studies of early relapsing remitting MS (Reindl et al. (1999) Brain, 122:2047-2056). A recently published study analyzed the prognostic significance of these antibodies in patients presenting with CIS, using a longitudinal design. This supports our assertion that antibody measurements can be used for prognosis in CIS patients, and in particular for predicting conversion to a diagnosis of clinical MS (Berger et al. (2003) N. Engl. J. Med, 349:139-135).

ELISA systems for specific detection of anti-Galc antibodies have been developed for marmosets and humans, and are routine in the laboratory. Extensive studies of reactivity to Galc have not yet been performed. Preliminary results indicate that anti-Galc antibodies are present in 67% of our SPMS cohort, and only 1 of the CIS patients studied, which suggests that these antibodies are associated with late and severe forms of MS.

5. Time course of autoreactive immune responses in MS.

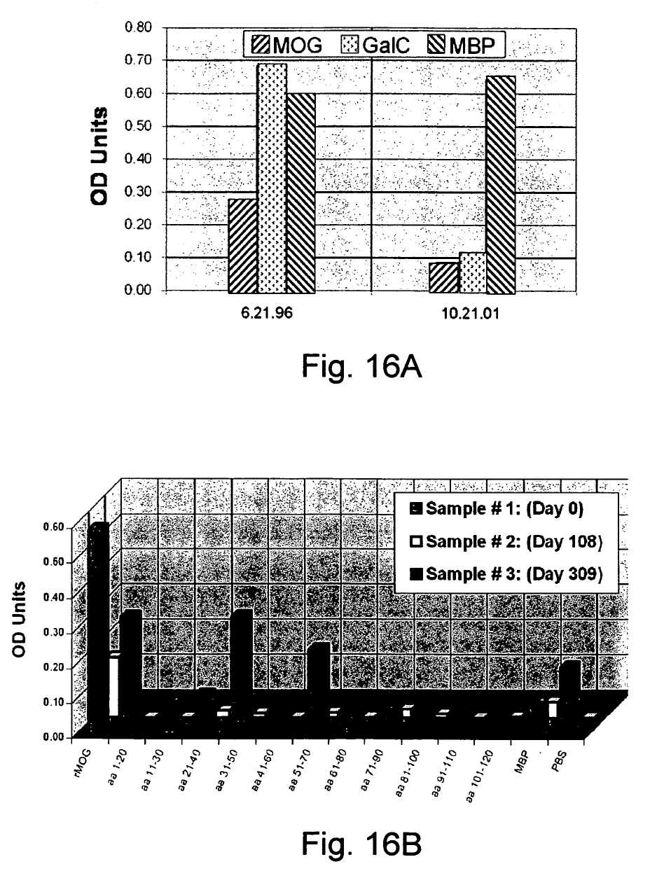

Previous work suggests that T cell autoreactivity to myelin antigens may vary in time, or may remain stable in given individuals. With the exception of some reports (Bielekova et al. (2000) Nature Medicine, 10:1167-1175), there is generally no obvious correlation between reactivity and occurrence of MS relapses (Pender et al. (2000) J. Immunol., 165(9):5322-5331; Tuohy et al. (1998) Immunol. Rev., 164:93-100; Goebels et al. (2000) Brain 123 Pt 3:508-518; Meinl et al. (1993) J Clin Invest., 92(6):2633-43; Hellings et al. (2002) J Neuroimmunol., 126(1-2):143-460; Lovett-Racke et al. (1997) J Neuroimmunol., 78(1-2):162-171). The time-dependency of autoantibody responses has not been systematically investigated in CNS demyelinating disorders, and available data are derived from cross-sectional studies (Reindl et al. (1999) Brain, 122:2047-2056). We had the opportunity to study serum samples from a few patients on repeated occasions for anti-myelin antibody reactivity. One patient (treated with interferonβ 1-a), showed a pronounced decrease in serum reactivity to MOG, and Galc over the course of 5 years (both IgG and IgM), and had no attack during this period. No change in anti-MBP antibody titers was observed, indicative that the change in MOG reactivity was not related to overall immune down-regulation. A second patient sampled on 3 occasions within a period of one year, similarly displayed time-dependent variability in MOG-specific IgG titers and peptide reactivity (FIG. 16). These preliminary results are consistent with studies of antibody reactivity in other autoimmune disorders showing that antibody reactivity may fluctuate in time. According to a recent review of diagnostic criteria, detection of antibodies upon two phlebotomies at 6 weeks interval is required for diagnosis of anti-phospholipid syndrome (Wilson et al. (1999) Arthritis Rheum., 42(7):1309-1311).

Methods

1. Analysis of Repertoire of Antibodies Against MOG

Sera from the CIS and MS patients tested for reactivity to MOGₐₐ₁₋₁₂₅ (rMOG), overlapping MOG peptides, MBP, and control antigens are studied using ELISA systems already developed in the laboratory. An aliquot of CSF is also included in these analyses, where a lumbar is required for clinical care of the patients. For individuals who display reactivity to MOG or MOG peptides (IgG and/or IgM) by ELISA, autoantibodies will be further characterized as follows:

1.1. Separation of conformation dependent and non-conformation dependent Ig fractions.

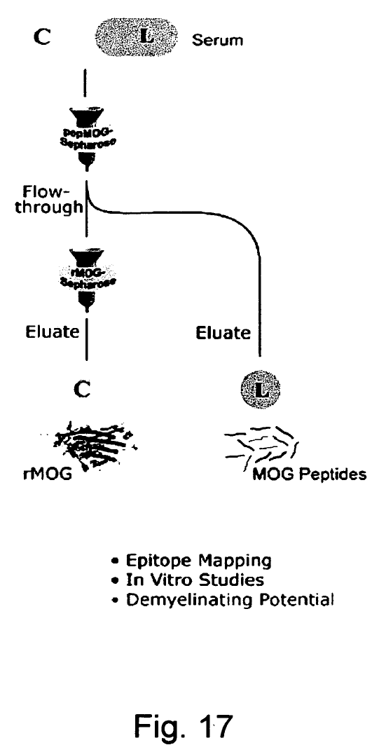

The MOG-reactive fraction in sera from MOG-seropositive patients (all Igs) are depleted from the peptide-reactive fractions by a pass on Sepharose columns coupled with MOG-peptides, and further purified on human rMOG-Sepharose affinity columns (FIG. 17). For the preparation of MOG-Sepharose affinity columns with the desired specificity, 200 μg of human rMOG, or MOG-derived 20 mer peptides (200 μg each) is reacted with NHS-Sepharose pre-packed in 1 ml columns, following the manufacturers instructions (Amersham Pharmacia). The column is ready for use after inactivation of unreacted NHS groups and washing. Serum is slowly loaded and, after extensive washing (PBS), bound antibody is eluted in buffer at pH 2.2 and immediately neutralized by addition of Tris buffer.

This protocol permits the isolation of conformation-binding (designated “C”) and linear peptide-binding antibody (“L”) fractions. Fractions are analyzed by SDS-PAGE/Western blotting and ELISA to confirm purity and antigenic specificity and Ig class. A second pass on the columns may be necessary to achieve >95% purity

1.2. In vitro binding studies.

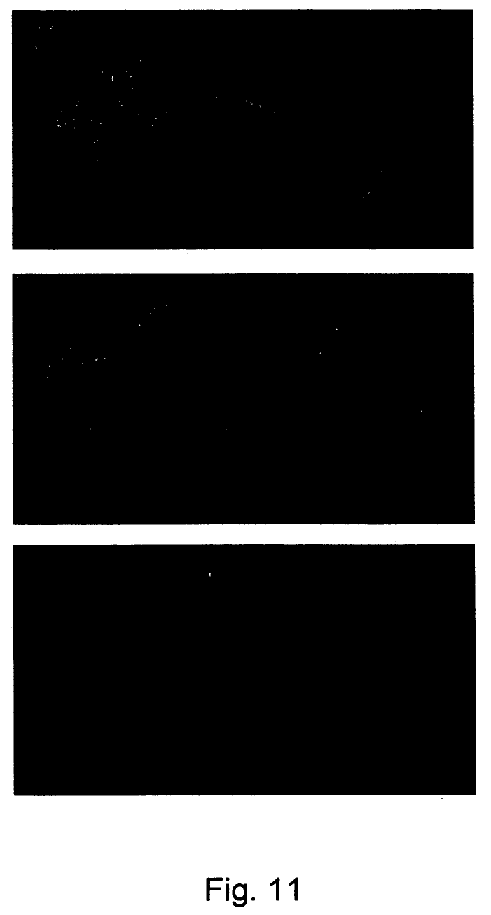

A pre-requisite for pathogenicity is that antibodies be capable of binding to exposed epitopes of MOG in situ on CNS myelin. To test this property of antibody fractions, we use flow cytometry. A human fibroblast cell line (CCL-153), COS cells, and a human oligodendroglioma cell line have been stably transfected with the human MOG gene cloned in a tetracycline-regulated expression vector (see, e.g., FIG. 11). A similar method with a mouse fibroblast transfected cell line has been successfully employed to characterize the conformational binding specificities of murine monoclonal anti-MOG antibodies (Brehm et al. (1999) J. Neuroimmunol., 97:9-15). Surface binding of Igs ua measured by flow cytometry on MOG-expressing cells and control, untransfected cells. Cells are washed and blocked with 3% normal goat serum, then incubated with the purified, biotinylated human Igs (using a commercial biotinylation kit), or unlabeled Igs and protein A/G-biotin. Fluorescence is detected using fluorescent-labeled streptavidin. Additional controls are performed in each experiment using an irrelevant Ig, or protein A/G-biotin, in the absence of Ig.

1.3. Competition between human antibodies and recombinant C. jacchus Fab.

The ability of affinity purified, MOG-specific human antibodies to compete with the monoclonal Fab fragments that define the epitope specificities of demyelinating C. jacchus antibody responsesare assessed in competition experiments using ELISA plates coated with rMOG. Fab fragments that represent structural epitope specificities isolated from C. jacchus, are expressed in soluble form and purified over protein L-affinity columns. The purity of these preparationsis confirmed by PAGE (single band at ˜55 kDa depending of the Fab, and reducing to L and truncated H chain under reducing conditions). Increasing concentrations of unlabeled, MOG-specific Fabs are incubated in the presence of affinity-purified Ig fractions. Bound Ig is detected using an Fc-specific, conjugated anti-human IgG antibody (see, e.g., Section C, FIG. 5).

2. General Material and Methods.

Human MOGaal-118, ratMOGaa1-125, rat MOGaa1-117 were produced in a transformed E. Coli strain available in the laboratory. The plasmid encodes for residues aa₁₋₁₂₅, with additional residues derived from the vector (MRGS at the N-ter) and a RSQSHHHHHH-(SEQ ID NO: 13) tag for affinity purification at the C-ter. rMOG is purified Ni-NTA-agarose columns using a standard protocol, yielding highly pure MOG as ascertained by SDS PAGE (major band at 15.9 kDa and a very minor band at ˜32 kDa corresponding to a dimer).

Native human MBP purified by the method of Deibler (Deibler et al. (1972) Prep. Biochem., 2:139-165) is available for these studies.

Galactocerebroside (1-0-Galactosyl-N-Acetyl-Sphingosine, C48H93NO8) from bovine spinal cord purified (>98%) by thin layer chromatography is purchased from commercial sources.

Panels of synthetic peptides are available as follows: MOG peptides, 20 mer overlapping peptides overlapping the sequence of the extracellular, Ig-like domain of MOG (aa1-120). A panel of 96 overlapping peptides (15 mers offset 3 and 12 mers offset 1 for immunodominant epitopes in marmosets and humans) encompassing the same domain of MOG, and several peptides located in the transmembrane regions of the protein that have recently been shown to be potential targets for MOG-directed T cell responses (Weissert et al. (2002) J. Immunol., 169(1):548-556). Synthetic MBP peptides are also available to extend these if needed.

ELISA systems.

Sera are separated from blood, properly aliquoted for analysis and antibody fractionation, and stored at −80° C. until use.

1. Human ELISA for MOG, MOG-peptides, and MBP:

These ELISAs are routine in the laboratory. Maxisorp plates are coated with 100 μl of 1 ug/ml antigen, washed and blocked with 3% bovine serum albumin. Serum is added at 3 dilutions. Second antibody is AP-labeled anti-IgG (Fc-specific), or anti-IgM (both 1:5,000), and color is developed with pNPP and read at 405 nm.

2. ELISA for Galc:

This ELISA is adapted from previously published studies (Ichioka et al. (1988) Neurochem Res., 13(3):203-207). Galc is sonicated and heated at 65° C. for 10 min and plated at a concentration of 5% on polystyrene ELISA plates (100 mcl/well). After blocking, 1:100 to 1: 1,000 dilutions of sera are added and incubated for 1 hr. at 37° C. Secondary antibody is anti-human IgG (Fc portion), 1:6,000, labeled with PE. The technique is identical to standard ELISAs with protein antigens, except that Tween is omitted from washes. Color development is performed by adding TMB substrate, and plates are read at 450 nm. Positive control is provided by a delipidized whole rabbit antiserum directed against Galc.

3. Quality control and quantitative measurements of antibody concentrations.

Standard curves: titers and actual concentrations of autoantibodies are obtained routinely. Standard curves are constructed using serial amounts of purified human IgG and included on each ELISA plate. Three serum dilutions are analyzed in duplicates, in order to establish an accurate determination of concentration. ELISA readings are analyzed in semi-quantitative (dilution titer) and quantitative (concentration) fashion. Criteria for positivity are: titer equal or greater than 1:100, concordant duplicate measurements, and signal greater than twice the background, with background less than 0.150 OD. The methods currently established in the laboratory detect IgG and IgM in separate assays, due to differences in processing and background for these individual Ig subtypes. A method for simultaneous detection of IgG and IgM is in development.

In addition to usual negative and positive control wells for antigen and secondary antibody, each assay can include control antigens (candida, measles, and/or tetanus toxoid), and negative and positive reference sera that have each been aliquoted in frozen single use vials. These assay systems show <1% intrassay and <5% interassay variability. An ongoing protocol conducts regular analysis of myelin and other antibody reactivity by ELISA in a separate cohort of control subjects (n=10, non MS), in order to control for other sources of variability (such as seasonal infections, for example).

### CExample 3

### The Ratio Of MOG-Peptide-Specific Over Rmog-Specific Antibodies Is Predictive Of The Severity Of Clinical EAE

FIG. 18 demonstrates that the ratio of MOG-peptide-specific over rMOG-specific antibodies is predictive of the severity of clinical EAE in the marmoset. Thus it appears to be an extremely useful index for evaluating MS patients.

As illustrated in FIG. 8, it is not possible to distinguish these different antibody fractions by ELISA or other standard antibody detection methods. The difference in epitope recognition may translate into functional heterogeneity (e.g., pathogenic potential), since marmosets immunized with the linear peptides develop an attenuated EAE phenotype compared to rMOG-immunized animals, despite the apparent induction of similar T cell responses. We have also observed that disease severity in rMOG-induced marmoset EAE is inversely proportional to the ratio of serum concentrations (μg/ml) of MOG peptide/rMOG-reactive IgG.

### Example 4

### Pathogenic properties of rMOG- and MOG peptide-specific antibodies

Passive antibody transfer experiments have been done in marmosets immunized with MBP that received homologous marmoset affinity-purified Ig fractions, either rMOG-specific (2 animals), rMOG-specific depleted from linear peptide-specificities (hence only conformation-dependent, 2 animals), and MOG peptide-specific (linear 20 mer peptides, 2 animals). Neuropathological examination obtained after antibody transfer revealed the presence of intra-parenchymal perivascular infiltrates with demyelination in all cases. However, reminiscent of the differences between rMOG-induced and MOG peptide induced EAE in marmosets, both the lesion burden and the extent of demyelination were much more pronounced in recipients of conformation dependent or rMOG-specific antibodies compared to animals receiving MOG peptide-specific antibodies (see FIG. 19).

These experiments provide direct confirmation that conformational antibodies and linear peptide-specific antibodies, although both recognizing rMOG, have strikingly different pathogenic potential. Although limited, the demyelination present in recipients of MOG peptide-specific Ig appears to be more severe than what has previously been observed in MBP-induced EAE or in MBP-immunized marmosets receiving control IgG, which have never been observed to develop pathology beyond rare subpial inflammatory infiltrates. These findings suggest that the MOG peptide-specific antibodies could also be pathogenic.

### Example 5

### Epitope recognition on the MOG protein differentially influences antibody effector functions and disease phenotype in autoimmune demyelination

In C. jacchus marmosets, immunization with myelin/oligodendrocyte glycoprotein (MOG, rMOG₁₋₁₂₅) produces a disseminated and demyelinating, multiple sclerosis (MS) like form of experimental allergic encephalomyelitis (EAE), with antibody responses against conformational and linear epitopes of MOG. By comparison, fewer, focal lesions mostly confined to subpial tissue of spinal cord and brainstem, less demyelination and autoantibodies that strictly target linear epitopes, characterize EAE induced with short MOG-derived peptides. To understand the basis for these phenotypic differences, we characterized effector mechanisms of pathogenicity associated with anti-MOG antibody subpopulations. Both linear and conformation-dependent antibodies were capable of binding to MOG in situ on myelin sheaths as demonstrated by immunohistochemistry. However, while macrophage/microglial activation was observed in both forms of EAE, IgG deposition and complement activation were only observed in lesions of rMOG₁₋₁₂₅immune marmosets. These findings indicate that polyclonal anti-MOG antibody repertoires in primates are highly heterogeneous in terms of pathogenicity, and that epitope recognition is a determinant factor of antibody effector functions and spatial dissemination of inflammatory demyelinating disease. Because marmoset and human anti-MOG repertoires target identical epitopes, this information offers critical insight for understanding the significance of antibody responses frequently detected in MS.

Introduction

Myelin/oligodendrocyte glycoprotein (MOG)-induced experimental allergic encephalomyelitis (EAE) in the common marmoset (C. jacchus) is a multifocal disease of central nervous system (CNS) white matter that closely approximates human multiple sclerosis (MS) (1-3). Myelin-directed T cell reactivity is obligatory for disease development in marmosets as in all EAE models, however involvement of anti-MOG antibodies is necessary for development of the typical MS-like neuropathological phenotype (4). Sensitization of rodents with immunodominant peptides of MOG gives rise to restricted antibody responses and usually suffices to induce severe EAE. Not unexpectedly however, a broader heterogeneity of epitopes within MOG antibody responses is found in higher mammals. Preliminary work suggests that diversity is present within the antiMOG antibody repertoire in humans (5, 6) and primates (7) and may underlie certain differences in the biological properties of autoantibodies (8). However, the relationship between anti-MOG antibody specificity and effector functions remains largely unexplored.

Structurally, antibodies against MOG can be differentiated on the basis of their ability to recognize either linear or conformational, tertiary structure-dependent epitopes (8). There is limited information on the respective pathogenicity of these antibody subgroups, which may differ depending on the species studied. Preliminary observations of marmoset EAE suggest that immunization with linear 20 mer MOG-derived peptides induces a form of EAE which is clearly different from that typically induced in this species by immunization with the entire extracellular domain of MOG (aa1-125, rMOG₁₋₁₂₅) (7, 8). Despite these discrepancies, standard ELISA methods detect antibody-reactivity against rMOG₁₋₁₂₅ and against linear MOG-epitopes at similar titers in both forms of MOGinduced marmoset EAE, as is also the case for rodents ^{9-11.} Thus, pathogenic properties and effector functions of the different antibody subtypes cannot be understood unless these antibody populations are isolated and separately studied. Such information is needed to facilitate the interpretation of findings of anti-MOG antibody reactivity in man, which in MS and control subjects has been reported with varying frequencies depending on the study, the method of detection and the form of MOG antigen used (6, 12, 13).

The recent cloning of recombinant MOG-reactive antibodies present in the marmoset immune repertoire has revealed monoclonal antibody specificities that define several distinct conformational, surface exposed epitopes of MOG, which are also present in the human antibody repertoires ^{8.} We have therefore taken advantage of this model system to characterize the immunopathogenicity of anti-MOG antibodies according to their epitope recognition. First we confirmed the association existing between presence of circulating antibodies with linear or structural anti-MOG specificity and neuropathological features of disease in a large series of animals immunized with linear MOG-derived peptides in comparison with the stereotyped pathology encountered in rMOG₁₋₁₂₅EAE ^{14.} We consistently found markedly reduced burden and dissemination of demyelinating lesions in these MOG peptide-immune animals despite the fact that MOG peptide-immune animals could clearly proceed to develop severe lethal EAE associated with large solitary destructive lesions. Second, we demonstrate that purified antibodies that recognize either conformational or linear epitopes are both capable of binding to MOG in situ in normal marmoset white matter. Third, the pathogenic functions of these different antibodies were elucidated by immunohistochemical characterization of inflammatory infiltrates. Lesion pathogenesis in MOG peptide-induced EAE appeared strikingly different from rMOG₁₋₁₂₅-induced disease. IgG deposition and complement activation were only observed in the latter with concomitant presence of antibodies against conformational determinants of MOG. However, both types of lesions were characterized by prominent macrophage infiltration. These findings are the first comprehensive analysis of the pathogenicity of polyclonal, native MOG antibodies populations representative of specificities that can be found in an outbred species. We formally demonstrate that while both linearly and conformationally defined antibodies may contribute to macrophage recruitment and activation, complement mediated demyelination is exclusively linked to in situ deposition of conformation-dependent antibodies. This information is crucial to the interpretation of MOG antibody responses in MS in context of the heterogeneity of pathology observed for this disease.

Materials and Methods

Antigens

A recombinant protein corresponding to the sequence of the extracellular domain of rat MOG (rMOG₁₋₁₂₅) was expressed and purified to homogeneity as fusion protein with a HiS₆-Tag in E. coli following published procedures (15). A panel of 11 synthetic overlapping linear 20 mer peptides corresponding to the sequence of the extracellular domain of rat MOG (aa1-120), and the C-terminus peptide of rMOG1-125 (WINPGRSRSHHHHHH (SEQ ID NO:______)) were synthesized using standard solid phase chemistry (Research Genetics, Huntsville, Ala.) and purified >95% by HPLC. Purity was confirmed by mass spectrometry.

Animals, Immunization and Characterization of EAE

C. jacchus marmosets used in this study were maintained in a primate colony at the University of California, San Francisco and were cared for in accordance with all guidelines of the Institutional Animal Care and Usage Committee (IACUC). Marmosets were actively immunized with either 50 μg of rMOG₁₋₁₂₅ (Group I), or 100 μg of MOG-derived 20 mer peptides (Group II, individual peptides or combinations, please also refer to Table 5) dissolved in phosphate buffered saline and emulsified with complete Freund's adjuvant (CFA) as previously described (1). The peptides, or combinations of peptides were selected according to previous mapping studies that have characterized the immunodominant T cell and antibody epitopes of rMOG₁₋₁₂₅ in marmosets (14, 16)

EAE was assessed by daily clinical examination and animals were observed for a total of 12 to 140 days (marmoset expanded scale, score 0 to 45 (17). At the end of the observation period, euthanasia was performed under deep pentobarbital anesthesia by intracardial perfusion with 4% para-formaldehyde, and the entire neuraxis obtained and examined in serial consecutive sections (2 mm each). Five μm, paraffin-embedded sections were stained with Luxol Fast Blue/Periodic Acid Schiff (LFB/PAS) or used for immunohistochemical analysis. Pathologic findings were graded according to separate inflammation and demyelination scores: Inflammation score: 0, no inflammation present; +, rare (1-3) inflammatory infiltrates/average whole section; ++, moderate numbers (310) of inflammatory infiltrates/section; +++, widespread parenchymal infiltration by inflammatory cells, with numerous large confluent lesions. Demyelination score: 0, no demyelination; +, rare (1-3 lesions/section) foci of demyelination; ++, moderate (3-10 lesions/section) demyelination; +++, extensive demyelination with large confluent lesions.

TABLE 5
Characteristics of EAE in groups I (rMOG₁₋₁₂₅-animals U004-99, U009-99,
Cj72-88, J2-97) and II (MOG peptide)-immune marmosets. pepMOG denotes a
mixture of 11 20 mer peptides overlapping by 10 amino acids (aa) and
spanning the sequence of MOG aa1-120.

| | | Max. clinical | | | |
|---|---|---|---|---|---|
| Animal ID | Immunogen | score | No. of lesions | Inflammation | Demyelination |
| U004-99 | rMOG₁₋₁₂₅ | 14 | 326 | +++ | +++ |
| U009-99 | rMOG₁₋₁₂₅ | 8 | 135 | +++ | +++ |
| Cj72-88 | rMOG₁₋₁₂₅ | 5.5 | 67 | +++ | +++ |
| J2-97 | rMOG₁₋₁₂₅ | 29 | 124 | +++ | +++ |
| 199-94 | aa21-40 | 8 | 4 | +++ | + |
| 368-94 | aa21-40 | 10 | 4 | ++ | + |
| 39-95 | pepMOG | 19 | 4 | +++ | + |
| 65-92 | pepMOG | 9 | 33 | ++ | +^{a} |
| 252-93 | aa1-40 | 5 | 6 | ++ | + |
| TX245-90 | aa1-40 | 10 | 8 | +++ | + |
| 14-91 | aa21-40, 51-90 | 12 | 8 | + | + |
| TX75-92 | aa51-90 | 10 | 13 | ++ | + |
| Tx256-93 | aa81-120 | 9 | 3 | + | + |

^{a}Demyelination was found with the grade indicated in all lesions except in animal 65-92, in which only 18 of 33 (55%) lesions were demyelinated.

Fractionation and purification of antibodies from immune C. jacchus sera

Sera were collected from each animal at euthanasia, and stored at −20° C. until use. The respective fractions of serum antibodies with binding specificities for linear peptide or conformational epitopes were separated by affinity chromatography. Sera or pools of sera from animals in groups I and II were repeatedly passed over columns containing a mixture of the 11 20 mer overlapping peptides spanning MOGaa1-120 (pepMOG) covalently linked to sepharose. Bound material containing the MOG peptide-reactive fraction (anti-MOG-P) was eluted with glycine buffer pH 2.5, immediately brought to neutral pH with 1 M Tris buffer (pH 8.0) and extensively dialyzed against PBS. Thus, in these experiments antibody reactivity found in flowthrough fractions (if present) could not represent any epitope of MOG directed against a linear feature, and was considered to represent conformation-dependent MOG-epitopes (anti-MOG-C). The binding characteristics of all eluted and flowthrough fractions were analyzed by ELISA. AntiMOG-C if present were further affinity-purified by passing pepMOG column flowthrough fractions over sepharose columns containing covalently linked rMOG₁₋₁₂₅, followed by elution, neutralization and dialysis as described above. In addition to characterization of fine specificity by ELISA, the ability of purified anti-MOG-P and anti-MOG-C to bind to native marmoset MOG in situ was determined by immunohistochemistry as described below using antibody fractions biotinylated with a sulfo-NHS biotinylation reagent following the manufacturers instruction (Pierce). Unreacted sulfo-NHS biotin was removed by extensive dialysis against PBS.

Epitope specificity

Epitope specificities of whole unfractionated sera, fractionated sera, or affinity-purified antibodies were determined by ELISA. Plastic wells (Pierce, Maleic Anhydride plates) were coated with rMOG₁₋₁₂₅ or MOG-derived 20 mer peptides. Control wells contained no antigen, the recombinant glutathione-S-transferase (GST) from E. Coli, and the (HiS)₆ C-terminal peptide of rMOG₁₋₁₂₅. Wells were blocked with PBS containing 0.05% Tween20 (PBS-T) and 3% bovine serum albumin (BSA), and the following samples were added in blocking buffer and incubated for 1 hour at 37° C.: 1. whole immune serum, 1:200; 2. Three μg/ml of affinity purified anti-MOG-P antibodies; 3. Group I _{(rMOG1-125-)}, or Group II (MOG peptide-immune) sera depleted of anti-MOG-P antibodies, 1:200. Next, a horseradish peroxidase labeled anti-monkey IgG (A0170, Sigma) was added, and after incubation for 1 hour, wells were developed with tetramethylbenzidine (TMB, Pierce) and read at 450 nm.

Immunohistochemistry

Sections of C. jacchus brain were de-paraffinized, hydrated, and treated with a citratebased antigen unmasking solution (Vector Labs, Burlingame, Calif.) at high temperature for 20 minutes. Endogenous peroxidase activity was blocked by incubation of sections in 0.3% _{H202} in methanol for 30 minutes. Sections were blocked with 5% normal goat serum (Sigma, St. Louis, Mo.) in PBS-T or 5% for 1 hour at 37° C., washed with PBS-T, and incubated with the following primary antibodies in blocking buffer: 1. Mouse anti-human C9neo (IgG1, Novocastra; 1:25) for staining of the terminal membrane attack complex (MAC); 2. mouse anti-human HAM56 (IgM, Accurate Chemicals; 1:20), panmacrophage/microglia marker; 3. mouse anti-human IgG (IgM, DAKO; 1::25). After incubation for 1 hour at 37° C. and washes with PBS-T, the appropriate biotinylated secondary antibodies were applied and incubated for another hour at 37° C. (rabbit antimouse IgG1 (Zymed); goat anti-mouse IgM (Vector)). Slides were rinsed again, incubated with the Vectastain Elite ABC Kit (Vector) and stained with 3,3′diaminobenzidine (DAB, Vector). All slides were counterstained with hematoxylin and permanently mounted. Biotinylated anti-MOG-P (from rMOG1-125-and MOG peptide-immune animals; 7 μg/ml and 20 μg/ml resp.) and anti-MOG-C (10 μg/ml) were used to characterize their ability to bind to native, full length MOG expressed in situ by oligodendrocytes in marmoset CNS.

T cell-proliferative responses

Peripheral blood mononuclear cells (PBMC) were isolated from blood samples obtained at euthanasia by centrifugation over a Ficoll gradient, and rested overnight in AIM-V media (Invitrogen). 1×10⁵ PBMC/well were incubated in triplicates in the presence of 10 μg/ml antigen (rMOG1-125, individual MOG-derived peptides) or without antigen (negative control) in 200 μl AIM-V and pulsed with 0.5 μCi ³H-thymidine after 48 hours. After an additional 18 hours, wells were harvested and ³H-thymidine incorporation was measured in a beta-counter. The stimulation-indices (S. I.) were calculated as the ratio of stimulated/control wells.

Statistics

The following quantitative and qualitative parameters were analyzed for each animal of groups I (n=4) and II (n=9): 1. Total lesion load by counting the number of lesions in 2024 sections stained with LFB covering the entire neuraxis. 2. Immunohistochemical patterns of staining by examining 2-4 sections of brain and spinal cord from group I (n=4, 24-82 lesions per animal), and all the sections containing lesions from animals in group II. We compared the percentages of lesions positive for each marker, with positivity defined as follows: HAM56, >10 stained cells/lesion; IgG (cellular distribution, B cells), >2 stained cells/lesion in the immediate perivascular vicinity; IgG (parenchymal distribution), clearly positive staining above background along fibers within lesions and not associated with cells; C9neo, clearly positive staining above background. Comparisons between the two groups were performed using a two-tailed, un-paired Student's t-test.

Results

Clinical and neuropathological characteristics of MOG peptide- and rMOG₁₋₁₂₅-induced EAE

All animals in the study developed clinical EAE, with variable severity observed in both groups I and II. Table 5 recapitulates the clinical and neuropathological phenotypes of EAE induced with the various immunogens. The course of MOG peptide-induced EAE tended to be progressive over time, with more rapid progression in the 2 animals immunized with pepMOG (the mixture of all peptides, #39-95 and 6592). rMOG1-125-induced EAE was either rapidly progressive or relapsing-remitting (not shown), as previously described in animals observed chronically. It is noteworthy that overall severity of disease was not associated with a particular immunization regimen: both animals J2-97 (rMOG₁₋₁₂₅-immune) and 39-95 (pepMOG-immune) developed hyperacute EAE symptoms requiring immediate euthanasia.

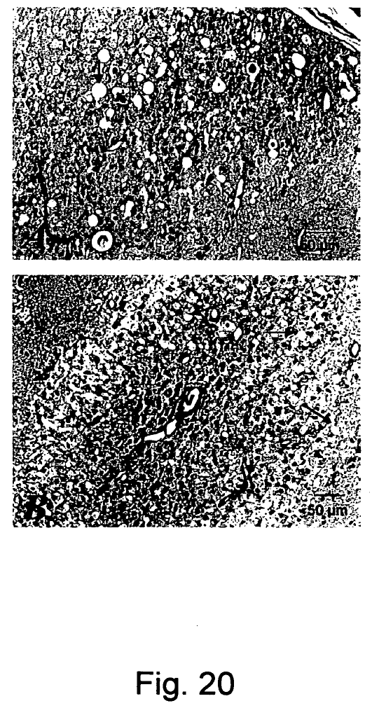

Neuropathologically, the most remarkable difference between animals in the 2 immunization groups was a tremendously reduced white matter lesion burden in group II (Table 5, FIG. 7: group I: 163 +/−56.3 lesions (mean +/−SEM); group II: 9.2 +/3.1, p=0.0012). Second, in contrast with the multifocal disease that we and others have consistently observed in many rMOG₁₋₁₂₅-immune marmosets (typically involving optic nerves, spinal cord, brain hemispheres, and brainstem with perivascular intraparenchymal distribution ¹⁴), the distribution of lesions in MOG peptide-immune animals was mainly restricted to brainstem and spinal cord with a pattern of subpial space infiltration reminiscent of MOG-peptide-induced EAE in mice (18, 19) (FIG. 20).

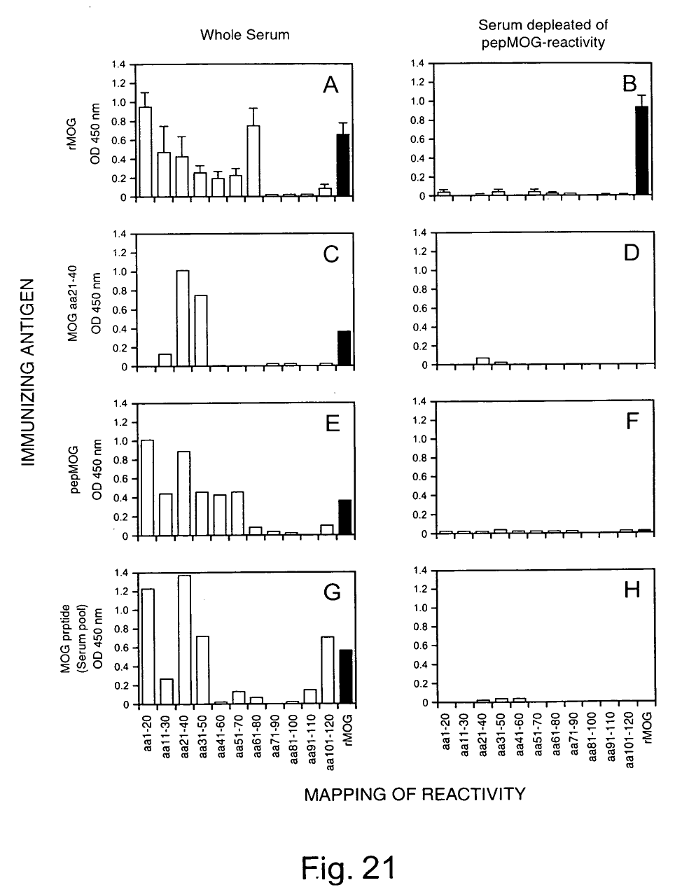

Animal 39-95 developed a large hemorrhagic lesion in the left optic tract and nerve. Only one of the pepMOG-immunized animals developed inflammatory lesions within the cerebral white matter (#65-92), none of which showed evidence of demyelination (not shown). The third major neuropathological difference between the two groups was that the extent of demyelinated areas was reduced in lesions of MOG peptide-induced EAE compared with those of rMOG₁₋₁₂₅-EAE, in the presence of roughly similar degrees of inflammation in most animals (Table 5). The demyelination in MOG peptide-induced EAE did not extend beyond the margin of inflammatory infiltrates, in contrast to the protracted and expanding lesions of rMOG₁₋₁₂₅-induced EAE (FIGS. 4 and 21). The most abundant pattern of demyelination in lesions of MOG peptide-induced EAE was myelin vacuolation, a feature that is present at the periphery of expanding lesions in rMOG1-125-induced marmoset EAE (3) (FIG. 20A).

Epitope specificities of antibody responses

As expected from previous studies, all monkeys developed serum antibodies that reacted to both rMOG₁₋₁₂₅ and linear peptides as shown by standard ELISAs of unfractionated serum (FIG. 21, left panels). To separately characterize conformational and linear specificities, the following selected sera were depleted from pepMOG-reactive antibodies: group I (rMOG₁₋₁₂₅-immune): J2-97, 72-88, U004-99, U009-99; group II: pepMOG-immune: 39-95, 65-92; MOG aa21-40-immune: 199-94, 368-94; a pool of equal amounts of sera from animals 14-91, 75-92, 252-93, 245-90, 256-93. The results from representative animals and the pooled sera from group II are shown in the right panels of FIG. 21.

Complete depletion of sera from anti-MOG-P antibodies was achieved after 3-5 passes over the pepMOG columns, as shown by the lack of binding to individual peptides (FIG. 21, right panels). Depletion from anti-MOG-P-resulted in complete loss of reactivity to rMOG₁₋₁₂₅ in each of the animals immunized with MOG peptides (FIG. 21D, F, H), regardless of the sequence of the immunizing peptides. By contrast, sera from rMOG₁₋₁₂₅₋ immune animals always retained reactivity against whole rMOG₁₋₁₂₅ after being depleted from anti-MOG-P antibodies (FIG. 21B).

Anti-MOG-P and anti-MOG-C antibodies from animals of both groups were eluted from the respective affinity columns. Only anti-MOG-P displayed binding to MOG peptides, as did the respective sera from which they were purified. These antibody fractions were also capable of binding to rMOG1-125 in vitro in the ELISA system (not shown).

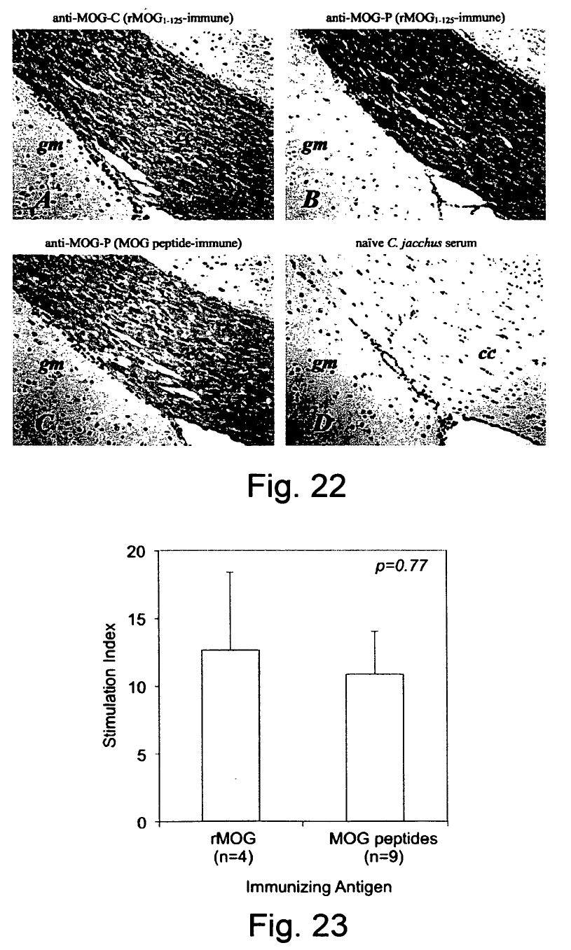

Incubation of normal marmoset CNS sections with anti-MOG-P antibodies showed that these antibodies strongly stained white matter, as did anti-MOG-C antibodies (FIG. 22). This was observed regardless of the immunization regimen used to produce anti-MOG-P antibodies (e.g., rMOG1-125 or MOG peptides) (FIG. 22). No significant reactivity to either recombinant GST expressed in E. coli or the (HiS)₆ C-terminal peptide was detected in any of the antibody fractions (not shown). Together, these findings demonstrated that: 1. Both linearly defined (anti-MOG-P) and conformational (anti-MOG-C) antibodies are capable of binding to MOG in situ, thus epitope recognition per se does not appear to be the determining factor for antibody binding to MOG embedded in intact myelin sheaths. 2. Anti-MOG-C antibodies that were isolated after depletion of pepMOG-specific antibodies were not directed against the C-terminal peptide of rMOG₁₋₁₂₅ or against bacterial contaminants in the rMOG₁₋₁₂₅-preparation used to synthesize the columns for affinity-chromatography.

MOG-specific T cell proliferative responses

Circulating T cell proliferative responses to rMOG₁₋₁₂₅ were observed in PBMC of all animals at euthanasia. The magnitude of these responses was similar in MOG peptideimmune animals and rMOG1-125-immune animals (10 +/−3.1 vs. 12.7 +/−5.8, NS, FIG. 23). T cell proliferative responses mapped to 20 mer peptides corresponding either to the immunodominant T cell epitopes in rMOG1-125-immune marmosets 7 or to the immunizing peptide(s) in MOG peptide-immune animals (not shown).

Immunohistochemical characterization of lesions

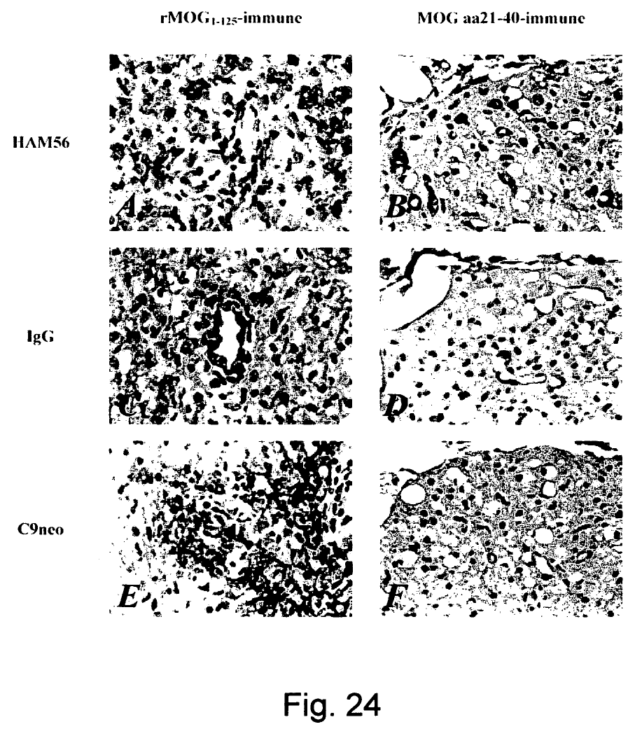

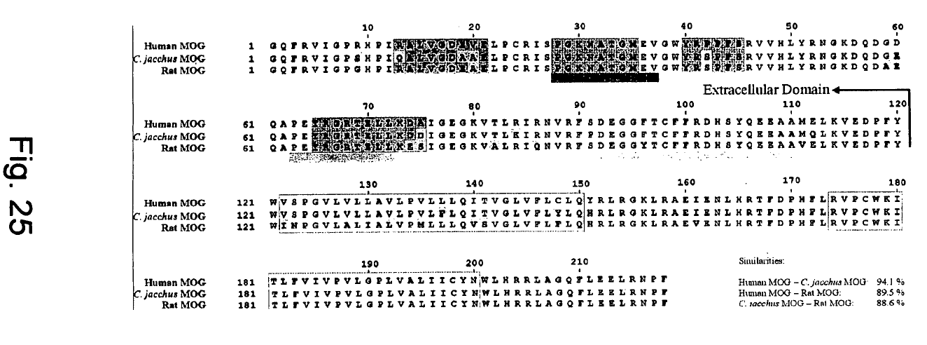

Results of immunostaining experiments are summarized in FIGS. 24 and 9. Macrophage infiltration was a consistent feature of inflammatory infiltrates in all animals, as indicated by staining for HAM56 (FIG. 24A+B). Pronounced IgG deposition was found in rMOG₁₁₂₅-immune animals, either in the immediate perivascular vicinity or deeper within the white matter parenchyma (FIG. 24C), in agreement with previous findings (20). In sharp contrast, lesions that showed IgG deposition were observed in only 2 animals immunized with MOG-peptides (39-95 and one in 252-93). This involved a single hemorrhagic lesion in both cases (not shown), which raises the possibility that this was the result of exsudation of blood into the lesion. In addition to parenchymal deposition, IgG could also be detected in cells present in close vicinity of blood vessels in rMOG₁₋₁₂₅-immune animals (B cells or plasmocytes, FIG. 24C). Some of the lesions found in MOG peptide-immune animals also showed IgG positive cells, though much less frequently (not shown). Quantitatively, the differences in IgG deposition and IgG positive cells between rMOG₁₋₁₂₅- and MOG peptide-immune animals were significant (p=0.003 and p=0.038 respectively, FIG. 9). Highly significant differences were also observed for C9neo deposition, which was prominently observed in rMOG1-125-immune animals (56% positive of 204 analyzed lesions), but was uniformly absent from any of the 83 lesions analyzed in MOG peptide-immune animals (0%, p<0.0001, FIG. 24E, F, and FIG. 9).

Discussion

T cell responses directed against one or several immunodominant linear peptides of MOG have been demonstrated to be powerful inducers of CNS inflammation and, in some EAE models, demyelination. The humoral responses against this encephalitogen however, appear to be much more complex in terms of determinant recognition and participation in lesion pathogenesis. The respective pathogenic potentials of antibodies directed against either linear or conformational determinants of MOG are not firmly established in all EAE models, and have not been investigated in primate species which share with humans the most complex antibody responses. Some studies of the murine anti-MOG antibody repertoire _{19, 21-23}, suggest that the recognition of conformational determinants of MOG may be an important requirement for pathogenicity. However, according to some ²⁴ but not all ¹¹ investigations, Lewis rats immunized with MOG aa35-55 can develop multifocal demyelinating disease, despite the demonstration that these animals do not develop conformation-dependent anti-MOG antibodies. This implies that MOG peptidespecific antibodies may be pathogenic in the rat, as seems to be the case in several mouse strains (10, 25, 26).

Regardless of these apparent species-specific differences, a role for humoral mechanisms of demyelination in human MS is even less clear than in EAE, although suggested by the presence of intrathecal immunoglobulin (Ig) synthesis (27) clonal expansion of B cells ₂₈₋₃₀ and complement activation ³¹. Anti-MOG antibody deposition has been recently demonstrated in context of a characteristic pattern of myelin disintegration in actively demyelinating lesions of both MS and marmoset EAE (2, 3). Despite one in vitro study that provides clues to the nature of exposed determinants of MOG in humans (5)′ to what extent the recognition of conformational determinants of MOG by B cells and/or antibodies influences the expression of MS phenotypes in humans remains largely unknown. We therefore designed the current studies to systematically investigate functional properties of anti-MOG antibodies according to epitope recognition in an outbred model of EAE that closely approximates the diversity of humoral responses and neuropathology encountered in MS (2, 3).

Mapping studies of anti-MOG antibody responses conducted in primate EAE and MS have generally paralleled these of T cell reactivity, using short peptides derived from MOG (5, 6, 9, 32, 33). As shown here and by others ^{24,} antibodies that are directed against linear epitopes can also bind to whole protein antigens, however standard techniques of antibody measurement do not discriminate between these antibodies and those that exclusively recognize conformational determinants of target antigens. We fractionated sera by affinity-chromatography to ensure complete distinction between antibody responses against strictly conformational and linear MOG epitopes, and demonstrate that marmosets immunized with MOG-derived peptides develop a restricted population of antibodies against the linear sequences. These animals fail to develop antibodies that are exclusively conformation-dependent, in contrast to rMOG₁₋₁₂₅-immunized marmosets. We took advantage of this finding to formally demonstrate a link between antibody epitope recognition and differential expression of EAE phenotypes.

It is important to note that the differences between rMOG₁₋₁₂₅-and MOG peptide-EAE predominantly involved patterns of disease dissemination and demyelination, and not severity of EAE. Animals in both groups developed either severe, rapidly progressive disease or mild to moderate forms, as can be expected in this outbred species. However, MOG peptide-immunized animals showed reduced disease burden and reduced, albeit significant demyelination compared to rMOG₁₋₁₂₅-immune animals. Demyelinating lesions in the former animals were mostly observed in spinal cord and brain stem, and not in cerebral hemispheres where they typically occur after rMOG₁₋₁₂₅-immunization _{7, 8} This pattern of pathology was a consistent feature of marmoset MOG peptide-EAE regardless of the choice of immunizing peptide within the extracellular domain of MOG, likely indicating that the observed differences were not a consequence of T cell epitope immunodominance. Rather, we propose that the recognition of conformational determinants of MOG was the basis for certain pathogenic properties of antibodies and/or B cells, which together with T cell responses resulted in MS-like multifocal and prominent demyelination.

The subpial localization of demyelinating infiltrates in MOG peptides-immunized marmosets is strikingly similar to CNS pathology observed in C57/B16 mice immunized with MOG peptide 35-55 (18, 19, 34). Our findings are in partial agreement with studies of mice lacking B cells, which fail to develop EAE after immunization with rMOG₁₋₁₂₀ (19)., Passive transfer of whole serum from wild type mice in these animals indicates that the MOG aa35-55 peptide-immune serum is less efficient than rMOG1-120-immune serum in restoring the EAE phenotype of wild type mice immunized with rMOG₁₋₁₂₀ (23). This could mean that the rMOG1-120 immune mouse serum contained certain pathogenic antibodies that are not present in MOG aa35-55-immune animals, as is the case for marmosets, however this was not investigated. In addition, these rodent studies have not addressed the question of disease dissemination, a major finding of the current work which clearly shows a link between antibody determinant recognition and density and distribution of CNS lesions. The presence of conformation dependent antibodies (anti-MOG-C) appears strictly associated with disease in a typical MS-like distribution (brain hemispheres, optic nerve and spinal cord), whereas linear-dependent antibodies are clearly associated with focal disease mostly restricted to brain stem and spinal cord in most animals. Possible biological explanations for these differences include differential binding affinity, or as discussed below different effector functions of anti-MOG-P and anti-MOG-C antibodies. It is also possible that the density of expression of MOG molecules, and/or presentation of its accessible epitopes on myelin sheaths differ within the different parts of the CNS, thus influencing lesion dissemination and location.

Certain immunopathological similarities appear to exist between MOG peptide- and rMOG₁₋₁₂₅-induced EAE. The lesion pattern observed in the former includes evidence of myelin vacuolation, which is also present at the edge of lesions in rMOG₁₋₁₂₅-induced EAE (3). This phenomenon has previously been shown to result for example, from exposure of the intact myelin sheath to a variety of toxic soluble substances such as TNFα (35) and triethyl tin sulfate (36) and could also be an effect of MOG-specific antibodies. Thus, a pathogenic role cannot be ruled out for anti-MOG-P antibodies that are induced in marmosets by immunization with either rMOG1-125 or MOG-derived peptides (see also discussion below on macrophage activation). Antibodies specific for MOG aa21-40 have been detected in close association with disintegrating myelin membranes in lesions of rMOG₁₋₁₂₅-induced marmoset EAE (2, 3), thus it is possible that anti-MOG-P antibodies play a pathogenic role in sustaining myelin-destruction by binding to epitopes newly exposed during active demyelination. Future studies of passive transfer of anti-MOG-P or anti-MOG-C in MBP sensitized animals should unequivocally determine which antibodies are capable of initiating certain patterns of demyelination.

Rat rMOG1-125 is ˜90% homologous to C. jacchus MOG₁₋₁₂₅ (37) and is a well established encephalitogen in this species _{38.} rMOG₁₋₁₂₅ and native C. jacchus MOG share identical conformational antibody epitopes, as demonstrated by immunohistochemical studies of marmoset brain conducted with monoclonal conformation-dependent Fab-fragments directed against rMOG₁₋₁₂₅ (8). Both anti-MOG-P (from rMOG1-125- and MOG peptideimmune animals), and anti-MOG-C were able to recognize native MOG in situ in normal CNS white matter. It is thus highly unlikely that the differences in neuropathology observed in our animals were due to amino acid substitutions between marmoset and rat MOG. Similarly, we found that anti-MOG-C antibodies (which were purified by complete removal of any linear specificity within MOG aa1-120 followed by purification on rMOG1-125 columns), did not bind to the (His)₆-tagged C-terminal portion of MOG₁₋₁₂₅, which is not present in native MOG expressed in marmoset CNS white matter.

Whether or not anti-MOG-P and anti-MOG-C bind to myelin with different affinities in vivo, parenchymal IgG deposition was only observed in rMOG₁₋₁₂₅-immunized marmosets, while it was noteworthy that macrophage infiltration and activation was present to a similar extent in both MOG peptide- and rMOG1-125-induced EAE. It is well known that macrophage infiltration and microglial activation can occur independently of stimulation by IgG via Fcγ-receptors, for example through activation by inflammatory mediators such as interferon-γ. Nevertheless, the recognition of structural epitopes of MOG not only could influence antibody binding in vivo but could also result in different effector mechanisms for antibody pathogenicity. Our results demonstrate that the C9 component of the lytic complex is only detected in brain tissue from rMOG-1-125immunized animals, and is absent from lesions of MOG peptide-induced EAE. This suggests that antibodies against conformation-dependent MOG epitopes, and not those against the linear epitopes are capable of activating lytic complement pathways, thus potentially augmenting the destructive potential of these antibodies. Numerous studies of EAE _{22, 39, 40} and MS _{31, 41} support a role for complement in lesion pathogenesis, however there is no practical marker to detect this type of pathology. The knowledge of what antibodies within the MOG repertoire have specific pathogenic properties is of considerable importance for future studies of humoral autoimmunity in MS.

Another intriguing finding of the current study was the absence of epitope-spreading to structure-dependent MOG-determinants in animals immunized with MOG-derived peptides. This suggests that exposure of complex, discontinuous determinants to B cells did not occur de novo in the context of myelin destruction in these animals, at least during an observation period of up to 140 days. These considerations have important implications for understanding molecular mimicry triggered by pathogens in terms of their capacity to induce pathogenic antibody responses. T cell mimicry between microbial peptides and myelin-antigens has been demonstrated in many experimental systems and can occur via direct sequence homology _{42,43} or bystander mechanisms (44). The current study provides strong evidence that humoral immunity against conformational determinants of MOG is a major modifying factor that could be responsible for disease dissemination within the CNS. Therefore, the presence of complex structural determinants similar to those of MOG would be required to provide the basis for molecular mimicry leading to such pathogenic antibody responses.

In summary, we present here the first comprehensive analysis of functional heterogeneity of the MOG-specific antibody repertoire in an outbred species that share complexity and similar structural epitopes with humans (8). The notorious heterogeneity of clinical presentations of MS has stimulated recent investigations demonstrating distinct neuropathological subtypes, some of which clearly involve humoral mechanisms of tissue damage ^{41.} We now demonstrate that antibody responses to a single target molecule of myelin are sharply dichotomized in terms of pathogenic and functional properties. Our observations bear important implications for the interpretation of anti-MOG antibody serotypes in humans and will be essential to guide the choice of future therapies antagonizing pathogenic antibody responses in MS.

### REFERENCES

1. Genain C P, Hauser S L. Creation of a model for multiple sclerosis in Callithrix jacchus marmosets. J Mol Med. 1997;75:187-197.
2. Genain C P, Cannella B, Hauser S L, Raine C S. Identification of autoantibodies associated with myelin damage in multiple sclerosis. Nat Med. 1999;5: 170-175.
3. Raine C S, Cannella B, Hauser S L, Genain C P. Demyelination in primate autoimmune encephalomyelitis and acute multiple sclerosis lesions: a case for antigen-specific antibody mediation. Ann Neurol. 1999;46: 144-160.
4. Genain C P, Nguyen M H, Letvin N L et al. Antibody facilitation of multiple sclerosis-like lesions in a nonhuman primate. J Clin Invest. 1995;96:2966-2974.
5. Haase C G, Guggenmos J, Brehm U et al. The fine specificity of the myelin oligodendrocyte glycoprotein autoantibody response in patients with multiple sclerosis and normal healthy controls. J Neuroimmunol. 2001; 114:220-225.
6. Lindert R B, Haase C G, Brehm U et al. Multiple sclerosis: B- and T-cell responses to the extracellular domain of the myelin oligodendrocyte glycoprotein. Brain. 1999;122:2089-2100.
7. von Büdingen H -C, Tanuma N, Villoslada P et al. Immune Responses Against The Myelin/Oligodendrocyte Glycoprotein In Experimental Autoimmune Demyelination. J Clin Immuol. 2001;21:155-170.
8. von Büdingen H C, Hauser S L, Fuhrmann A et al. Molecular characterization of antibody specificities against myelin/oligodendrocyte glycoprotein in autoimmune demyelination. Proc Natl Acad Sci USA. 2002;99:8207-8212.
9. Kerlero de Rosbo N, Brok H P, Bauer J et al. Rhesus monkeys are highly susceptible to experimental autoimmune encephalomyelitis induced by myelin oligodendrocyte glycoprotein: characterisation of immunodominant T- and B-cell epitopes. J Neuroimmunol. 2000;110:83-96.
10. Bernard C C, Johns T G, Slavin A et al. Myelin oligodendrocyte glycoprotein: a novel candidate autoantigen in multiple sclerosis. J Mol Med. 1997;75:77-88.
11. Adelmann M, Wood J, Benzel I et al. The N-terminal domain of the myelin oligodendrocyte glycoprotein (MOG) induces acute demyelinating experimental autoimmune encephalomyelitis in the Lewis rat. J Neuroimmunol. 1995;63: 17-27.
12. Xiao B G, Linington C, Link H. Antibodies to myelin-oligodendrocyte glycoprotein in cerebrospinal fluid from patients with multiple sclerosis and controls. J Neuroimmunol. 1991;31:91-96.
13. Karni A, Bakimer-Kleiner R, Abramsky O, Ben-Nun A. Elevated levels of antibody to myelin oligodendrocyte glycoprotein is not specific for patients with multiple sclerosis. Arch Neurol. 1999;56:311-315.
14. Genain C P, Hauser S L. Experimental allergic encephalomyelitis in the New World monkey Callithrix jacchus. Immunol Rev. 2001;183:159-172.
15. Amor S, Groome N, Linington C et al. Identification of epitopes of myelin oligodendrocyte glycoprotein for the induction of experimental allergic encephalomyelitis in SJL and Biozzi AB/H mice. J. Immunol. 1994;153:43494356.
16. Brok H, Uccelli A, Kerlero De Rosbo N et al. Myelin/oligodendrocyte glycoprotein-induced autoimmune encephalomyelitis in common marmosets: the encephalitogenic T cell epitope pMOG24-36 is presented by a monomorphic MHC class II molecule. J. Immunol. 2000; 165:1093-1101.
17. Villoslada P, Hauser S L, Bartke I et al. Human nerve growth factor protects common marmosets against autoimmune encephalomyelitis by switching the balance of T helper cell type 1 and 2 cytokines within the central nervous system. J Exp Med. 2000;191:1799-1806.
18. Hjelmstrom P, Juedes A E, Fjell J, Ruddle N H. B-cell-deficient mice develop experimental allergic encephalomyelitis with demyelination after myelin oligodendrocyte glycoprotein sensitization. J. Immunol. 1998;161:4480-4483.
19. Lyons J A, San M, Happ M P, Cross A H. B cells are critical to induction of experimental allergic encephalomyelitis by protein but not by a short encephalitogenic peptide. Eur J. Immunol. 1999;29:3432-3439.
20. McFarland H I, Lobito A A, Johnson MM et al. Determinant spreading associated with demyelination in a nonhuman primate model of multiple sclerosis. J. Immunol. 1999;162:2384-2390.
21. Bourquin C, Iglesias A, Berger T et al. Myelin oligodendrocyte glycoprotein-DNA vaccination induces antibody-mediated autoaggression in experimental autoimmune encephalomyelitis. Eur J. Immunol. 2000;30:3663-3671.
22. Brehm U, Piddlesden S J, Gardinier M V, Linington C. Epitope specificity of demyelinating monoclonal autoantibodies directed against the human myelin oligodendrocyte glycoprotein (MOG). J Neuroimmunol. 1999;97:9-15.
23. Lyons J A, Ramsbottom M J, Cross A H. Critical role of antigen-specific antibody in experimental autoimmune encephalomyelitis induced by recombinant myelin oligodendrocyte glycoprotein. Eur J. Immunol. 2002;32:1905-1913.
24. Ichikawa M, Johns T G, Adelmann M, Bernard C C. Antibody response in Lewis rats injected with myelin oligodendrocyte glycoprotein derived peptides. Int Immunol. 1996;8: 1667-1674.
25. Slavin A J, Johns T G, Orian J M, Bernard C C. Regulation of myelin oligodendrocyte glycoprotein in different species throughout development. Dev Neurosci. 1997;19:69-78.
26. Albouz-Abo S, Wilson J C, Bernard C C, von Itzstein M. A conformational study of the human and rat encephalitogenic myelin oligodendrocyte glycoprotein peptides 35-55. Eur J Biochem. 1997;246:59-70.
27. Tourtellotte W W, Potvin A R, Fleming J O et al. Multiple sclerosis: measurement and validation of central nervous system IgG synthesis rate. Neurology. 1980;30: 240-244.
28. Baranzini S E, Jeong M C, Butunoi C et al. B cell repertoire diversity and clonal expansion in multiple sclerosis brain lesions. J. Immunol. 1999;163:5133-5144.
29. Colombo M, Dono M, Gazzola P et al. Accumulation of clonally related B lymphocytes in the cerebrospinal fluid of multiple sclerosis patients. J. Immunol. 2000;164:2782-2789.
30. Owens G P, Kraus H, Burgoon M P et al. Restricted use of VH4 germline segments in an acute multiple sclerosis brain. Ann Neurol. 1998;43:236-243.
31. Storch M K, Piddlesden S, Haltia M et al. Multiple sclerosis: in situ evidence for antibody- and complement-mediated demyelination. Ann Neurol. 1998;43:465471.
32. Genain C P, Abel K, Belmar N et al. Late complications of immune deviation therapy in a non human primate. Science. 1996;274:2054-2057.
33. Genain C, Belmar N, Diaz-Villoslada P, Hauser S. Fine Specificities of T cell and B cell responses to myelin/oligodendrocyte glycoprotein in common marmosets. J Neuroimmunol. 1998;90:34.
34. Svensson L, Abdul-Majid K B, Bauer J et al. A comparative analysis of B cellmediated myelin oligodendrocyte glycoprotein-experimental autoimmune encephalomyelitis pathogenesis in B cell-deficient mice reveals an effect on demyelination. Eur J. Immunol. 2002;32:1939-1946.
35. Selmaj K, Raine C S. Tumor necrosis factor mediates myelin damage in organotypic cultures of nervous tissue. Ann N Y Acad. Sci. 1988;540:568-570.
36. Aleu F P, Katzman R, Terry R D. Fine structure and electrolyte analyses of cerebral edema induced by alkyl tin intoxication. J Neuropathol Exp Neurol. 1963;22:403-413.
37. Mesleh M F, Belmar N, Lu C W et al. Marmoset Fine B Cell and T Cell Epitope Specificities Mapped onto a Homology Model of the Extracellular Domain of Human Myelin Oligodendrocyte Glycoprotein. Neurobiol Dis. 2002;9:160-172.
38. Genain C P, Hauser S L. Allergic Encephalomyelitis in Common Marmosets: Pathogenesis of a Multiple Sclerosis-like Lesion. Methods. 1996;10:420-434.
39. Piddlesden S, Lassmann H, Laffafian I et al. Antibody-mediated demyelination in experimental allergic encephalomyelitis is independent of complement membrane attack complex formation. Clin Exp Immunol. 1991;83:245-250.
40. Piddlesden S J, Lassmann H, Zimprich F et al. The demyelinating potential of antibodies to myelin oligodendrocyte glycoprotein is related to their ability to fix complement. Am J Pathol. 1993;143:555-564.
41. Lucchinetti C, Bruck W, Parisi J et al. Heterogeneity of multiple sclerosis lesions: implications for the pathogenesis of demyelination. Ann Neurol. 2000;47:707717.
42. Fujinami R S, Oldstone M B. Amino acid homology between the encephalitogenic site of myelin basic protein and virus: mechanism for autoimmunity. Science. 1985;230: 1043-1045.
43. Wucherpfennig K W, Strominger J L. Molecular mimicry in T cell-mediated autoimmunity: viral peptides activate human T cell clones specific for myelin basic protein. Cell. 1995;80:695-705.
44. Miller S D, Katz-Levy Y, Neville K L, Vanderlugt C L. Virus-induced autoimmunity: epitope spreading to myelin autoepitopes in Theiler's virus infection of the central nervous system. Adv Virus Res. 2001;56:199-217.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of diagnosing or evaluating the prognosis of multiple sclerosis (MS) or allergic encephalomyelitis (EAE) in a mammal, said method comprising:
detecting the presence or quantity of an antibody in said mammal specific for a conformational epitope of myelin/oligodendrocyte glycoprotein (MOG);
where the presence or increased concentration of said antibodies indicates the presence of a particular stage of multiple sclerosis or the increased likelihood of the development of a more severe form of the disease.

2. The method of claim 1, wherein said detecting comprises obtaining a biological sample comprising serum or cerebrospinal fluid from said mammal.

3. The method of claim 1, wherein said detecting comprises screening for a plurality of antibodies specific for different conformational epitopes of said myelin/oligodendrocyte glycoprotein.

4. The method of claim 1, wherein said antibody specific for a conformational epitope of myelin/oligodendrocyte glycoprotein is an antibody that specifically binds to an epitope specifically bound by an antibody comprising a polypeptide sequence selected from the group consisting of SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, and SEQ ID NO:37.

5. The method of claim 1, wherein said detecting comprises a competitive assay using a competitive binder an antibody comprising a CDR3 comprising a peptide sequence as shown in Table 2 (SEQ ID NOs:1-12).

6. The method of claim 1, wherein said detecting comprises a competitive assay using as a competitive binder an antibody specific for a conformational epitope of myelin/oligodendrocyte glycoprotein is an antibody that specifically binds to an epitope bound by an antibody comprising a polypeptide sequence selected from the group consisting of SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, and SEQ ID NO:37.

7. The method of claim 1, wherein said detecting comprises a competitive assay using as a competitive binder an antibody specific for a conformational epitope of myelin/oligodendrocyte glycoprotein where said antibody comprises a polypeptide sequence selected from the group consisting of SEQ ID NO:15, SEQ ID NO: 17, SEQ ID NO: 19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, and SEQ ID NO:37.

8. The method of claim 1, wherein said mammal is a human.

9. The method of claim 1, wherein said mammal is a human with a preliminary diagnosis of multiple sclerosis.

10. A method of evaluating the risk of progressing to a severe form of multiple sclerosis and/or the extent of central nervous system damage in a mammal, said method comprising:
obtaining a biological sample comprising serum or cerebrospinal fluid from said mammal; and
detecting the proportion of autoantibodies specific for a conformational epitope to those specific for a linear MOG epitope or a linear epitope of another myelin protein;
where an increased ratio of conformational specific antibodies indicates an increased likelihood or progressing to a severe form of the disease and/or increased central nervous system damage.

11. The method of claim 10, wherein detecting said proportion comprises detecting binding of autoantibodies to a MOG conformational epitope and to a MOG linear peptide.

12. The method of claim 10, wherein detecting said proportion comprises determining the ratio of MOG-peptide-specific to rMOG-specific antibodies.

13. The method of claim 10, wherein said detecting comprises screening for a plurality of antibodies specific for different conformational epitopes of said myelin/oligodendrocyte glycoprotein.

14. The method of claim 10, wherein the antibodies specific for a conformational epitope of myelin/oligodendrocyte glycoprotein include an antibody that specifically binds to an epitope bound by an antibody comprising a polypeptide sequence selected from the group consisting of SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, and SEQ ID NO:37.

15. The method of claim 10, wherein said detecting comprises a competitive assay using a competitive binder an antibody comprising a CDR3 comprising a peptide sequence as shown in Table 2 (SEQ ID NOs:1-12).

16. The method of claim 10, wherein said detecting comprises a competitive assay using as a competitive binder an antibody specific for a conformational epitope of myelin/oligodendrocyte glycoprotein is an antibody that specifically binds to an epitope bound by an antibody comprising a polypeptide sequence selected from the group consisting of SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, and SEQ ID NO:37.

17. The method of claim 10, wherein said detecting comprises a competitive assay using as a competitive binder an antibody specific for a conformational epitope of myelin/oligodendrocyte glycoprotein where said antibody comprises a polypeptide sequence selected from the group consisting of SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, and SEQ ID NO:37.

18. The method of claim 10, wherein said mammal is a human.

19. The method of claim 10, wherein said mammal is a human with a preliminary diagnosis of multiple sclerosis.

20. A method of treating a patient having a preliminary diagnosis of multiple sclerosis, said method comprising:
obtaining a biological sample comprising serum from said patient;
determining the ratio of autoantibodies specific for a conformational epitope to those specific for a linear MOG epitope or a linear epitope of another myelin protein; and
prescribing a more aggressive treatment regimen when said ratio is elevated.

21. A method of diagnosing definite multiple sclerosis in patients with a first episode of demyelination in the central nervous system, said method comprising:
measuring antibodies against specific myelin constituents;
where the presence of such antibodies indicates a definite diagnosis of multiple sclerosis.

22. The method of claim 21, wherein said myelin constituent comprises MOG.

23. The method of claim 21, wherein said myelin constituent comprises Galc.

24. The method of claim 21, wherein said antibodies are specific for a conformational epitope of MOG.

25. The method of claim 21, wherein said antibodies are specific for a conformational epitope of Galc.

26. A method of determining the form of multiple sclerosis, said method comprising:
measuring a plurality of antibodies against specific myelin constituents;
where presence or level of certain members of said plurality indicate the form or stage of multiple sclerosis.

27. The method of claim 26, wherein said myelin constituent comprises MOG.

28. The method of claim 26, wherein said myelin constituent comprises Galc.

29. The method of claim 26, wherein said detecting comprises
detecting the presence or quantity of an antibody specific for a conformational epitope of myelin/oligodendrocyte glycoprotein (MOG).

30. The method of claim 26, wherein said detecting comprises screening for a plurality of antibodies specific for different conformational epitopes of said myelin/oligodendrocyte glycoprotein.

31. The method of claim 30, wherein said antibody specific for a conformational epitope of myelin/oligodendrocyte glycoprotein is an antibody that specifically binds to an epitope bound by an antibody comprising a polypeptide sequence selected from the group consisting of SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, and SEQ ID NO:37.

32. The method of claim 26, wherein said detecting comprises a competitive assay using a competitive binder an antibody comprising a CDR3 comprising a peptide sequence as shown in Table 2 (SEQ ID NOs:1-12).

33. The method of claim 26, wherein said detecting comprises a competitive assay using as a competitive binder an antibody specific for a conformational epitope of myelin/oligodendrocyte glycoprotein is an antibody that specifically binds to an epitope bound by an antibody comprising a polypeptide sequence selected from the group consisting of SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, and SEQ ID NO:37.

34. The method of claim 26, wherein said detecting comprises a competitive assay using as a competitive binder an antibody specific for a conformational epitope of myelin/oligodendrocyte glycoprotein where said antibody comprises a polypeptide sequence selected from the group consisting of SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, and SEQ ID NO:37.

35. A method of predicting disease outcome in patients with a first episode of demyelination in the central nervous system or with definitive multiple sclerosis, said method comprising:
measuring antibodies against specific myelin constituents;
where the presence or increasing concentrations of such antibodies indicates a progressively negative outcome.

36. The method of claim 35, wherein said myelin constituent comprises MOG.

37. The method of claim 35, wherein said myelin constituent comprises Galc.

38. The method of claim 35, wherein said antibodies are specific for a conformational epitope of MOG.

39. The method of claim 35, wherein said antibodies are specific for a conformational epitope of Galc.

40. The method of claim 35 comprising measuring said antibodies at two or more times.

41. The method of claim 40, wherein said two or more times comprises a first time at initial presentation or diagnosis of said disease and a second time at least two months later.

42. A method of estimating the time within the history of an individual patient when MS disease will transform from benign to progressive, said method comprising:
measuring a plurality of antibodies against specific myelin constituents;
where presence or level of certain members of said plurality indicate the imminence of transformation of MS from benign form to a progressive form.

43. The method of claim 42, wherein said myelin constituent comprises MOG.

44. The method of claim 42, wherein said myelin constituent comprises Galc.

45. The method of claim 42, wherein said measuring comprises
detecting the presence or quantity of an antibody specific for a conformational epitope of myelin/oligodendrocyte glycoprotein (MOG).

46. The method of claim 42, wherein said measuring comprises screening for a plurality of antibodies specific for different conformational epitopes of said myelin/oligodendrocyte glycoprotein.

47. The method of claim 45, wherein said antibody specific for a conformational epitope of myelin/oligodendrocyte glycoprotein is an antibody that specifically binds to an epitope bound by an antibody comprising a polypeptide sequence selected from the group consisting of SEQ ID NO:15, SEQ ID NO:17, SEQ ID NO:19, SEQ ID NO:21, SEQ ID NO:23, SEQ ID NO:25, SEQ ID NO:27, SEQ ID NO:29, SEQ ID NO:31, SEQ ID NO:33, SEQ ID NO:35, and SEQ ID NO:37.

48. The method of claim 42 comprising measuring said antibodies at two or more times.

49. The method of claim 48, wherein said two or more times comprises a first time at initial presentation or diagnosis of said disease and a second time at least two months later.

50. A recombinant protein consisting of a MOG extracellular domain and a truncation at the C-terminus, wherein said protein is soluble in an aquous buffer at neutral pH.

51. The recombinant protein of claim 50, wherein said protein is a protein selected from the group consisting of Rat MOG 1-117, Rat MOG 1-125, human MOG 1-118, and human MOG 1-125.

52. An assay for detecting antibodies to conformational epitopes of MOG in a mammal, said assay comprising:
providing a serum or CSF sample from said subject; and
contacting antibodies in said sample with two or more recombinant proteins of 50;
where specific binding of one or more of said recombinant proteins to said antibodies indicates the presence of one or more antibodies antibodies to conformational epitopes of MOG in said mammal.

53. The method of claim 52, wherein said two or more proteins are independently selected from the group consisting of Rat MOG 1-117, Rat MOG 1-125, human MOG 1-118, and human MOG 1-125.
